(19) United States
(12) Patent Application Publication
(10) Pub. No.: US 2011/0262988 A1
(43) Pub. Date: Oct. 27, 2011

(54) METHODS AND COMPOSITIONS FOR ENHANCED BACTERIAL HYDROLYSIS OF CELLULOSIC FEEDSTOCKS

(75) Inventor: Ely Morag, Rehovot (IL)

(73) Assignee: DESIGNER ENERGY LTD., Rehovot (IL)

(21) Appl. No.: 13/129,507

(22) PCT Filed: Nov. 16, 2009
(86) PCT No.: PCT/IB2009/055083

§ 371 Date: May 16, 2011

Related U.S. Application Data
(60) Provisional application No. 61/115,303, filed on Nov. 17, 2008.

(51) Int. Cl. C12P 1/04; C07H 99/00; C12N 9/24

(52) U.S. Cl. 435/170; 435/200; 536/1.11

(57) ABSTRACT

The present invention provides compositions comprising a cellulolytic bacterium and an isolated BETA-glucosidase enzyme or comprising cellulolytic bacterium that expresses a secreted recombinant BETA-glucosidase enzyme, and methods of using same for hydrolysis of cellulose and cellulosic feedstocks.

### FIELD OF THE INVENTION

Embodiments of the present invention relate to the production of soluble carbohydrate from lignocellulosic and other types of cellulose-containing biomass material by a combination of native bacterial hydrolysis and supplementary enzymes. Other embodiments of the invention relate to methods for enhancing hydrolysis lignocellulosic biomass and use of these processes to produce fermentable sugars such as glucose.

### BACKGROUND

Sugar Production from Lignocellulosic Biomass

Bacterial and enzymatic conversion of cellulose-containing biomass into soluble sugars has wide application, including, inter alia, the production of ethanol for fuel applications which is currently commanding attention due to the increasing price of fossil fuel and environmental concerns. It will be noted that lignocellulosic biomass, as opposed to human and animal edible substrates such as sugarcane, does not affect food prices or availability and thus offers advantages. These factors, together with the ready availability of cellulosic waste and rising costs of edible substrates, have spurred increased worldwide research and developmental efforts by both governmental organizations and industrial entities.

Ethanol production from biomass, for example, typically includes three major steps: physicochemical pretreatment, enzymatic hydrolysis using cellulases, and fermentation of the soluble sugar into ethanol. Utilization of cellulase enzymes contributes significantly to the cost of production, typically being some 20-30% of the total cost. Current approaches to improving the cost-efficacy of this process include optimizing cellulase enzyme production, improving the stability and the activity of these enzymes, and reducing fermentation and purification costs (Zhang Y H et al, Ni J, et al).

An alternate approach is the use of microorganisms such as bacteria and fungi for direct and/or indirect bioconversion of lignocellulosic biomass into ethanol (Kubicek C P et al). This approach provides the advantage of incorporating all stages necessary for ethanol production into one or two simple and cost-effective processes. Currently, however, efficiencies of such processes are hampered by limited cellulosic degradation rates, low hydrolysis yields, and low maximal concentrations of lignocellulosic substrate, resulting in low yields of soluble sugar and ethanol.

### Clostridium Thermocellum and its Use in Cellulose Degradation

Cellulolytic, thermophilic anaerobic bacteria such as Clostridium thermocellum (Lynd et al) have been proposed as a means of generation of breaking down cellulosic biomass into sugars. C. thermocellum degrades cellulose, forming cellobiose and cellodextrins as the main products. Cellobiose, a disaccharide of two glucose moieties held together by a BETA-1,4 linkage, is imported and further hydrolyzed by the organisms, yielding ethanol, acetic acid, lactic acid, hydrogen, and carbon dioxide as the end products (Lamed et al). Small cellodextrins can also be taken into the cell, broken down further, and metabolized.

### BETA-Glucosidase Enzymes

The enzyme BETA-glucosidase, IUBMB Enzyme Nomenclature EC 3.2.1.21, and its enzymatic activity, hydrolysis of terminal, non-reducing β-D-glucosyl residues with release of β-D-glucose, have been known for years (Conchie, 1954). Among other substrates, BETA-glucosidase is capable of hydrolyzing cellobiose to form glucose.

### The C. Thermocellum Cellulosome

The C. thermocellum cellulase complex, or cellulosome, is an enzymatic scaffold that is secreted from the cell and/or displayed on the cell surface. The cellulosome can completely solubilize crystalline forms of cellulose such as cotton and Avicel, an activity known as “true cellulase activity” or “Avicelase activity”. The cellulosome contains: (i) endo-BETA-glucanase enzymes, which catabolize amorphous types of cellulose, including CMC and trinitrophenyl carboxymethylcellulose (TNP-CMC); (ii) exoglucanase enzymes, which cleave large, insoluble cellulose fragments into smaller, soluble cellodextrins; and (iii) a variety of exo xylanases and other carbohydrate hydrolyases. Products of cellulose degradation by the cellulosome are transported into the cell and further processed by cellobiose phosphorylase, which catabolizes cellobiose to glucose and glucose-1-phosphate; cellodextrin phosphorylase, which phosphorylates BETA-1,4-oligoglucans; and intracellular BETA-glucosidase enzymes, which hydrolyze cellobiose to glucose.

Cellobiose, a major product of cellulose degradation, is utilized by cellulolytic bacteria as a major carbon and energy source. Cellobiose is transported into the cell via an active transport system and hydrolyzed by several BETA-glucosidase enzymes to glucose and phosphoglucose.

BETA-glucosidase enzymes have been included in an enzyme cocktail added to non-cellulolytic organisms for use in production of ethanol. The enzyme cocktail is used to enable production of glucose from cellulosic materials, since such organisms are incapable of hydrolyzing cellulose overall and, in particular, do not possess a means of hydrolyzing cellobiose; the glucose is then used as a substrate for fermentation into ethanol (Kotaka et al).

BETA-glucosidase enzymes have been also added to purified cellulase complexes in in-vitro experimental models and found to enhance cellulase activity (Kosugi et al). This was attributed to inhibition of the cellulosome by cellobiose (Johnson et al), coupled with the fact that the purified cellulase complex is not able to hydrolyze or otherwise process cellobiose (Kadam et al).

### SUMMARY OF THE INVENTION

Certain embodiments of the present invention relate to methods for stimulating cellulolytic activity of intact bacteria by addition of exogenous BETA-glucosidase that functions outside the bacteria. Other embodiments of the present invention are related to a composition comprising at least one exogenous BETA-glucosidase enzyme and a cellulolytic microorganism. An isolated BETA-glucosidase enzyme, for example, may be added to the growth medium early in the cellulose hydrolysis process or/and after hydrolysis is underway. In certain embodiments, addition of the purified BETA-glucosidase significantly enhances the utilization of lignocellulosic biomass and accumulation of soluble fermentable sugars, mainly in the form of glucose and as cellobiose and xylose as well. In other embodiments, accumulation of soluble sugars is also significantly enhanced. Under the conditions utilized in the experiments described herein, microcrystalline cellulose (MC) utilization was enhanced by 50-100%, and soluble sugar content was increased by about 2-5 fold. In addition, under the conditions utilized, utilization of 60-70% of total MC was achieved, which compares favorably to 30-40% as achievable for the bacterium alone. The above-mentioned advantages have been also observed for a wide range of concentrations of MC. In addition, stimulation of bacterial growth on the insoluble substrate was indicated by a change in the color of the substrate during the hydrolysis process from white (native color) to pale-yellow, then to deep-yellow late in the hydrolysis process. The color change was associated with colonization of the bacteria on the cellulose and was particularly evident 12-24 hours after inoculation and is associated with acceleration of cellulose hydrolysis. These advantages were also observed with pretreated biomass. Thus, certain embodiments of the present invention exhibit advantages when compared to other direct and indirect bioconversion of cellulosic biomass into soluble sugars by cellulolytic microorganisms.

Cellulolytic bacteria are known to import cellobiose, to hydrolyze it to glucose and other degradation products using an intracellular BETA-glucosidase activity, and to utilize these products as an energy source. By contrast, external glucose cannot be utilized as a carbon and energy source by cellulolytic bacteria such as C. thermocellum, since they have no means of transporting glucose into the cell. External addition of BETA-glucosidase to intact cellulolytic bacteria was thus not expected to confer any advantage to their growth or ability to metabolize cellulosic biomass and rather was expected to be energetically unfavorable to bacteria. However, the present inventors have surprisingly found that addition of exogenous BETA-glucosidase enzymes enhances production of soluble carbohydrate from cellulose-containing biomass by cellulolytic organisms. Furthermore, bacterial growth on cellulosic substrates was found to be stimulated.

Other embodiments of the present invention relate to methods for stimulating cellulolytic activity of intact C. thermocellum bacteria by maintaining the pH of the medium at a level known to be preferred for bacterial growth and metabolism for a portion of the cellulose hydrolysis process, then removing the pH control at a defined point and allowing the hydrolysis process to continue, referred to herein as “two-stage pH control”. In the case of C. thermocellum, optimum proliferation occurs at pH 7-7.2. Within the range of 5-6.5, the catabolism and proliferation of the bacteria significantly slow down, so soluble sugars are not efficiently consumed. In contrast, the cellulolytic system of C. thermocellum works at an optimum pH of about 5. The present inventors have exploited these differences in pH for metabolism vs. biomass hydrolysis to increase the production of soluble sugars and the rate of hydrolysis.

### BRIEF DESCRIPTION OF THE DRAWINGS

The following figures are by way of illustrative example and are not meant to be taken as limiting the claimed invention.

FIG. 1. SDS-PAGE gel showing BETA-glucosidase expression in total cell extracts of BL1 (DE3) cells transfected with pET28a vector (lane 1) and in purified aliquots of the extracts (lanes 2-4).

FIG. 2. Graph showing accelerated bacterial hydrolysis in the presence of exogenous C. thermocellum BETA-glucosidase and dose response effect of BETA-glucosidase on hydrolysis of 8 gr/L microcrystalline cellulose (MC). Data for 0.3 ml and 0.6 ml groups are superimpose for first 3 timepoints.

FIG. 3. Graph showing accelerated cellulose hydrolysis in the presence of fungal BETA-glucosidase in a growth medium containing 21 gr/L MC.

FIG. 4. Graph showing the accumulation of reducing sugar from bacterial hydrolysis in the presence of fungal and bacterial BETA-glucosidase in growth medium containing 21 gr/L MC.

FIG. 5. TLC analysis showing reaction products of bacterial hydrolysis in the presence of fungal and bacterial BETA-glucosidase.

FIG. 6. Graph showing accelerated bacterial cellulose hydrolysis in the presence of BETA-glucosidase in growth medium containing 40 gr/L MC.

FIG. 7. Graph showing accumulation of reducing sugar in the absence or presence of added BETA-glucosidase in growth medium containing 40 gr/L MC.

FIG. 8. Graph showing accelerated bacterial cellulose hydrolysis in the presence of BETA-glucosidase in growth medium containing 80 gr/L MC.

FIG. 9. Graph showing further enhancement of bacterial cellulose hydrolysis by sequential addition of BETA-glucosidase. Times of administration of BETA-glucosidase are indicated by “BGL” underscored with two dots.

FIG. 10. Graph showing accumulation of reducing sugar following single or sequential addition of BETA-glucosidase or in the absence of BETA-glucosidase.

FIG. 11. Graph showing the effect of inoculum type on cellulose rate hydrolysis in the absence or presence of added BETA-glucosidase.

FIG. 12. Graph showing the effect of inoculum type on accumulation of reducing sugar in the absence or presence of added BETA-glucosidase.

FIG. 13. Graph showing yellow affinity substance accumulation in the absence or presence of added BETA-glucosidase.

FIG. 14. Graph depicting reducing sugar accumulation from bacterial hydrolysis of pretreated switchgrass in the absence or presence of exogenous BETA-glucosidase in a 1.3 Liter bioreactor in the absence of pH control.

FIG. 15. Graph depicting amount of residual biomass in the experiment described for FIG. 14.

FIG. 16. Graph depicting a comparison of two-stage pH control vs. no pH control as determined by amount of residual biomass.

FIG. 17. Graph depicting a comparison of two-stage pH control vs. no pH control as determined by reducing sugar accumulation.

### DETAILED DESCRIPTION OF THE EMBODIMENTS

Embodiments of the present invention relate to mixtures comprising a cellulolytic bacterium and (a) an isolated BETA-glucosidase enzyme and/or (b) a cellulolytic bacterium that expresses a secreted recombinant BETA-glucosidase enzyme, and methods of using same for hydrolysis of cellulose, cellulosic biomass, and cellulosic waste material.

As used herein, the term “recombinant enzyme” relates to an enzyme where the nucleic acid molecule that encodes the enzyme has been modified in vitro, so that its sequence is not naturally occurring, or is a naturally occurring sequence added to a genome in which it is not ordinarily present.

One embodiment of the present invention provides a mixture comprising a cellulolytic bacterium, a medium comprising a cellulosic feedstock, and an isolated BETA-glucosidase enzyme. The term “isolated BETA-glucosidase enzyme” as used herein refers to a BETA-glucosidase enzyme provided independently of the cellulolytic bacterium. The enzyme may be provided in any form known in the art, including, inter alia, as a powder, crystalline material or solution. In another embodiment, the isolated BETA-glucosidase enzyme is a recombinant BETA-glucosidase enzyme. In another embodiment, the BETA-glucosidase enzyme is purified from a natural source. Each possibility may be considered as being a separate embodiment of the present invention.

Another embodiment of the present invention provides a mixture comprising a cellulolytic bacterium and a medium comprising a cellulosic feedstock, wherein the cellulolytic bacterium has been engineered to produce an exogenous, secreted BETA-glucosidase enzyme, either using a leader peptide fused to the BETA-glucosidase or by another method. In light of the data provided herein, similar effects to those achieved using exogenous BETA-glucosidase enzymes, as described hereinbelow may be accomplished by engineering a cellulolytic bacterium to express a secreted BETA-glucosidase enzyme. Each possibility may be considered as being a separate embodiment of the present invention.

Another embodiment of the present invention provides a method of hydrolyzing a cellulosic feedstock, the method comprising the step of incubating a medium comprising the cellulosic feedstock with a cellulolytic bacterium and an isolated BETA-glucosidase enzyme, thereby hydrolyzing a cellulosic feedstock. In various embodiments, the BETA-glucosidase enzyme may be added prior to beginning the incubation step or at one or more times following commencement of the incubation step. Another embodiment of the present invention provides a method of producing ethanol, the method comprising the steps of hydrolyzing a cellulosic feedstock by the above method and allowing soluble sugars produced thereby to ferment into ethanol. Each possibility may be considered as being a separate embodiment of the present invention.

Another embodiment of the present invention provides a method of hydrolyzing a cellulosic feedstock, the method comprising the step of incubating a mixture of the present invention, the mixture comprising a cellulolytic bacterium engineered to express a soluble, exogenous BETA-glucosidase enzyme, thereby hydrolyzing cellulose. In another embodiment, the cellulolytic bacterium is engineered to display BETA-glucosidase on its cell-surface. Another embodiment of the present invention provides a method of producing ethanol, the method comprising the steps of hydrolyzing a cellulosic feedstock by the above method and allowing soluble sugars produced thereby to ferment into ethanol. Each possibility may be considered as being a separate embodiment of the present invention.

Optionally, a method of the present invention further comprises the step of adding an additional aliquot of the BETA-glucosidase enzyme to the mixture during the incubation at a time point subsequent to the first addition. In another embodiment, an additional aliquot of the BETA-glucosidase enzyme is added to the mixture at least at two time points during the incubation. In another embodiment, an additional aliquot is added at more than two time points during the incubation. In another embodiment, an additional aliquot is added at least at three time points during the incubation. In another embodiment, an additional aliquot is added at more than two three points during the incubation. In another embodiment, BETA-glucosidase enzyme is continuously added to the mixture using an external pump apparatus or other apparatus that continuously adds the enzyme. As provided herein, addition of BETA-glucosidase enzyme at multiple time points during a cellulose hydrolysis reaction provides an additional enhancement of hydrolysis. In certain embodiments, the additional enhancement is still greater when the pH is below a level consistent with bacterial replication and metabolism. Each possibility may be considered as being a separate embodiment of the present invention.

In another embodiment, aliquots of BETA-glucosidase enzyme are added at least once per 48 hours. In another embodiment, aliquots are added at least once per 72 hours. In another embodiment, aliquots are added at least once per 96 hours. In another embodiment, aliquots are added at least once per 24 hours. In another embodiment, aliquots are added at least twice within the first 24 hours. In another embodiment, aliquots are added at least twice within the first 48 hours. In another embodiment, aliquots are added at least twice within the first 72 hours. In another embodiment, aliquots are added at least twice within the first 96 hours. Each possibility may be considered as being a separate embodiment of the present invention.

Other embodiments of the present invention provide a product that has been produced by a method of the present invention or by a process utilizing a mixture of the present invention. In various embodiments, the product comprises a sugar, a mixture of sugars, and/or a fermentation product thereof. In certain embodiments, the fermentation product comprises ethanol. Each possibility may be considered as being a separate embodiment of the present invention.

In certain embodiments, the cellulose hydrolysis is performed in a container, inter alia a cellulose hydrolysis apparatus, capable of holding a liquid medium such as a liquid fermentation medium. In another embodiment, the incubation is performed under agitation. In another embodiment, the incubation is performed with constant agitation. In another embodiment, the incubation is performed for a time period sufficient to hydrolyze the desired amount of cellulose or cellulosic biomass. Each possibility may be considered as being a separate embodiment of the present invention.

The term “mixture” as used herein refers to a combination of the recited elements in any form known in the art, including, inter alia, liquid, solution, suspension, solid, or semi-solid form or a combination thereof.

The term “cellulose hydrolysis apparatus” as used herein refers to an apparatus suitable for a cellulose hydrolysis reaction. Containers for cellulose hydrolysis apparatuses useful in methods and compositions of the present invention include inter alia fermentors, serum bottles, shake flasks, and bioreactors. Various types of bioreactors may be useful in methods and compositions of the present invention, including inter alia, percolated impellor bioreactors, draught tube air-lift bioreactors, draft tube with lasplan turbine bioreactors, air-lift loop bioreactors, rotating drum bioreactors, and spin filter bioreactors.

As known to those skilled in the art, a bioreactor is a type of flask adapted/developed for fermentation under controlled conditions. Typically, a bioreactor is capable of controlling the pH, temperature, and/or oxygen saturation conditions of the medium inside the bioreactor. Bioreactors useful for methods and compositions of the present invention may include diagnostic mechanisms for measuring the pH and/or temperature conditions of the medium; mechanisms for adjusting one or more of the above parameters; and a mechanism for stirring or mixing the medium. Bioreactors are well known in the art, and are described, inter alia, in U.S. Pat. Nos. 7,604,987, 7,537,926, 5,512,480, 5,338,447, and 5,205,935, which are incorporated herein by reference. Each possibility may be considered as being a separate embodiment of the present invention.

According to some embodiments, the method of the present invention is performed in batch culture. According to some further embodiments, the method of the present invention is performed in fed-batch culture. According to some embodiments, the method of the present invention is performed in continuous culture. Each possibility may be considered as being a separate embodiment of the present invention.

As provided herein, the inventors have discovered that, in certain embodiments, addition to cellulolytic bacteria of an exogenous BETA-glucosidase enzyme exogenous to the bacteria significantly increases the rate and yield of hydrolysis of cellulosic feedstock and the amount of cellulose that can be utilized. Further, BETA-glucosidase stimulates accumulation of yellow affinity substance, consistent with enhanced bacterial growth.

In certain embodiments of the present invention, the temperature of the medium utilized in methods and compositions of the present invention is over 40° C. and the pH of the medium is within the range of 5.0-6.5. In certain other embodiments, the pH of the medium is below 6.5. In certain other embodiments, the pH of the medium is below 6.0. In certain other embodiments, the pH of the medium is between about 4.5 and about 6.5. In certain other embodiments, the pH of the medium is between about 5 and about 5.5. As provided herein, bacteria incubated under pH conditions not conducive to bacterial replication and metabolism exhibit an increase in cellulose hydrolysis relative to conditions conducive to bacterial replication and metabolism. Thus, under conditions conducive for cellulose hydrolysis, inter alia a temperature of 40-95° C. in the case of a thermophilic bacterium, hydrolysis is increased. In certain embodiments, the cellulose hydrolysis apparatus is a bioreactor, and the pH is not controlled. In other embodiments, the pH of the medium is lowered to a level below 5.0 by addition of an acidifying agent. Each possibility may be considered as being a separate embodiment of the present invention.

In other embodiments, the cellulose hydrolysis of methods of the present invention is performed in two stages, wherein: a. the pH of the medium is maintained at a value consistent with bacterial replication and metabolism during the first stage; and b. during the second stage, the pH is not maintained at a value consistent with bacterial replication and metabolism. During the second stage of hydrolysis, the pH is lowered to a level not consistent with bacterial replication and metabolism by either inactivating the pH-controlling mechanism and/or addition of an acidifying agent. In the latter case, the pH may gradually drop due to acidic metabolites secreted by the bacteria as a result of continued hydrolysis of cellulose. In the case of C. thermocellum, optimum proliferation occurs at pH 7-7.2, while optimum hydrolysis of cellulosic biomass occurs within the range of 5-6.5, As provided herein, such hydrolysis methods, referred to as “two-stage pH control” methods, provide in certain embodiments superior cellulose hydrolysis compared to either controlling pH during the entire hydrolysis reaction or not controlling pH during the entire hydrolysis reaction. In other embodiments, an additional enhancement is observed when two-stage pH control is combined with sequential BETA-glucosidase addition. Each possibility may be considered as being a separate embodiment of the present invention.

It will be understood by those skilled in the art that the exact point at which the pH controlling mechanism is inactivated is not critical to achieving the results presented herein. In certain embodiments, the pH controlling mechanism is removed at a defined point in the hydrolysis. The defined point can be inter alia a defined amount of base, a defined time, a defined bacterial density, or a combination thereof. In certain embodiments, under the conditions utilized herein, the pH controlling mechanism is removed after addition of about 5-20 ml of 4M NaOH or an equivalent amount of base per liter. In certain embodiments, under the conditions utilized herein, the pH controlling mechanism is removed between about 6-36 hours after commencing the hydrolysis reaction. In certain other embodiments, the pH controlling mechanism is removed using another criterion known to those skilled in the art. Each possibility may be considered as being a separate embodiment of the present invention.

Reference to a pH value “consistent with bacterial replication and metabolism” as used herein refers to a value wherein the bacterium utilized in the hydrolysis is able to reproduce at an appreciable rate and to consume soluble sugar generated by the hydrolysis of the biomass. In the case of C. thermocellum, this value may be between 6.5 and 7.5. In other embodiments, the value may be between pH 7-7.5. In other embodiments, the value may be about pH 7-7.2. In certain embodiments, the pH range is such that replication and/or metabolism rate is not reduced by more than 25% relative to the rate at the optimum pH level for replication and/or metabolism. In certain other embodiments, the replication and/or metabolism rate is not reduced by more than 50% relative to the rate at the optimum pH level. In certain other embodiments, the replication and/or metabolism rate is not reduced by more than 75% relative to the rate at the optimum pH level. During the second stage, the pH is reduced (either actively or passively) to a level not consistent with efficient bacterial replication and/or metabolism. In the case of C. thermocellum, this value may be below 6.5. In other embodiments, the value may be below 6.0. In other embodiments, the value may be between 5.5 and 6.5. In other embodiments, the value may be between 5.0 and 6.5. Each possibility may be considered as being a separate embodiment of the present invention.

The term “cellulosic feedstock” as used herein refers to a medium that contains cellulose as its major energy source. Various types of cellulosic biomass and cellulosic waste material comprise cellulose as their major energy source. Thus, the cellulosic feedstock of methods and compositions of the present invention is, in another embodiment, selected from a cellulosic biomass and a cellulosic waste material. The term “cellulosic biomass” as used herein refers to any treated or untreated natural cellulose-containing substance. Many sources of cellulosic biomass are known in the art. In another embodiment, the source of the cellulosic biomass is selected from the group consisting of switchgrass, corn-stover, corn straw, wheat straw, rice straw, Miscanthus x giganteus, poplar, wood chip, prairie grass, soft-wood, hard-wood, and bagasse. In another embodiment, the cellulosic biomass is any other cellulosic biomass known in the art. Each possibility may be considered as being a separate embodiment of the present invention.

In other embodiments, the major energy source of the medium utilized in methods and compositions of the present invention consists essentially of a cellulosic feedstock. In another embodiment, a cellulosic feedstock is the major energy source. In another embodiment, a cellulosic feedstock is the only significant energy source. In another embodiment, one or more other energy sources besides a cellulosic feedstock are also present. Each possibility may be considered as being a separate embodiment of the present invention.

The term “cellulosic waste material” as used herein refers to any cellulose-containing waste product of an industrial or agricultural process. Many sources of cellulosic waste are known in the art. In another embodiment, the cellulosic waste is selected from the group consisting of paper milling waste, recycled paper, and waste paper. In another embodiment, the cellulosic waste is another cellulosic waste known in the art. Each possibility may be considered as being a separate embodiment of the present invention.

In certain embodiments, a cellulosic feedstock may be dissolved and/or suspended in a growth medium utilized in methods and compositions of the present invention. In another embodiment, the medium has a cellulose content of at least 40 grams per liter (g/L). As provided herein, certain embodiments of mixtures and methods of the present invention enable utilization of cellulosic feedstocks containing cellulose in amounts of 40-80 g/L microcrystalline cellulose (MC) or an equivalent amount of cellulose in another form. In another embodiment, the amount is at least 60 g/L MC or an equivalent amount of another form. In another embodiment, the amount is at least 80 g/L MC or an equivalent amount of another form. In another embodiment, the amount is at least 100 g/L MC or an equivalent amount of another form. In another embodiment, the amount is at least 150 g/L MC or an equivalent amount of another form. In another embodiment, the amount is at least 200 g/L MC or an equivalent amount of another form. In another embodiment, the amount is at least 250 g/L MC or an equivalent amount of another form. In another embodiment, the amount is at least 300 g/L MC or an equivalent amount of another form. In another embodiment, the amount is at least 400 g/L MC or an equivalent amount of another form. In another embodiment, the amount is at least 440 g/L MC or an equivalent amount of another form. In another embodiment, the amount is 40-100 g/L MC or an equivalent amount of another form. In another embodiment, the amount is 40-200 g/L MC or an equivalent amount of another form. In another embodiment, the amount is 40-300 g/L MC or an equivalent amount of another form. In another embodiment, the amount is 40-400 g/L MC or an equivalent amount of another form. In another embodiment, the amount is 40-440 g/L MC or an equivalent amount of another form. In another embodiment, a medium of methods and compositions of the present invention contains at least one of the above amounts of purified cellulose. As provided herein, certain embodiments of methods and compositions of the present invention enable utilization of larger amounts of cellulose than methods lacking one or more features of the present invention. Each possibility may be considered as being a separate embodiment of the present invention.

In another embodiment, the cellulosic biomass of methods and compositions of the present invention has been pretreated to remove the lignin therefrom. In another embodiment, the cellulosic biomass of methods and compositions of the present invention has been pretreated to remove the hemicellulose therefrom. In another embodiment, the cellulosic biomass has been pretreated to reduce the lignin content. Pretreatment of biomass such as switchgrass increases the amount of biomass that can be utilized by cellulolytic bacteria.

The term “pretreated cellulosic waste or feedstock” as used herein refers to cellulosic waste or feedstock that has been treated to facilitate hydrolysis by cellulolytic bacteria. In another embodiment, the cellulosic waste or feedstock has been treated by milling. In another embodiment, the milling comprises one of the following methods: ball milling, two-roll milling, hammer milling, or vibro energy milling. In another embodiment, the pretreatment comprises irradiation. In another embodiment, the irradiation comprises gamma ray, electron-beam, or microwave irradiation. In another embodiment, the pretreatment comprises physical treatment. In another embodiment, the physical treatment comprises hydrothermal, high pressure, steam treatment, expansion, pyrolysis, or extrusion. In another embodiment, the pretreatment comprises explosive disruption. In another embodiment, the explosive disruption comprises a steam explosion, ammonia fiber/APEX-ammonia fiber explosion, CO₂ explosion, or SO₂ explosion. In another embodiment, the pretreatment comprises alkali treatment. In another embodiment, the alkali treatment comprises lime, sodium hydroxide, ammonia, ammonia sulfite, or mixtures thereof. In another embodiment, the pretreatment comprises acid treatment. In another embodiment, the acid treatment comprises sulfuric acid, hydrochloric acid, phosphoric acid, or mixtures thereof. In another embodiment, the pretreatment comprises gas treatment. In another embodiment, the gas treatment comprises chlorine dioxide, nitrogen dioxide, sulfur dioxide, or mixtures thereof. In another embodiment, the pretreatment comprises an oxidizing agent. In another embodiment, the oxidizing treatment comprises hydrogen, peroxide, wet oxidation, ozone, or mixtures thereof. In another embodiment, the pretreatment comprises solvent treatment. In another embodiment, the solvent treatment comprises ethanol-water extraction, benzene-water extraction, or butanol-water extraction swelling agents. Each possibility may be considered as being a separate embodiment of the present invention.

In another embodiment, a method of the present invention further comprises pretreatment using hypochlorite-containing carbonate buffer. Such buffers may be used, in certain embodiments, at room temperature, typically about 20-25° C. In other embodiments, a buffer containing 3-12% sodium hypochlorite is utilized. In other embodiments, a sodium hypochlorite-containing carbonate buffer is utilized. In certain non-limiting embodiments, such buffers may have a pH of 11-13. In certain other non-limiting embodiments, such buffers may be utilized at a liquid/solid ratio of between 0.4:1 and 2:1. In certain other non-limiting embodiments, such buffers may be utilized under constant agitation. In certain other non-limiting embodiments, such treatment is followed by a washing step to remove most or all of the hypochlorite. Each possibility may be considered as being a separate embodiment of the present invention.

In other embodiments, the medium utilized in methods and compositions of the present invention comprises a cellulosic biomass having a cellulose content of 40-80 g/L MC, which is equivalent to 80-200 gr/L of typical natural untreated cellulosic feedstock. This value, 80-200 gr/L, can further be increased after pretreatment to remove the ligin from the cellulosic feedstock. In another embodiment, cellulosic biomass is present in an amount of at least 100 g/L. In another embodiment, the amount is at least 120 g/L. In another embodiment, the amount is at least 140 g/L. In another embodiment, the amount is at least 160 g/L. In another embodiment, the amount is at least 180 g/L. In another embodiment, the amount is at least 200 g/L. In another embodiment, the amount is at least 220 g/L. In another embodiment, the amount is at least 250 g/L. In another embodiment, the amount is at least 300 g/L. In another embodiment, the amount is at least 350 g/L. In another embodiment, the amount is at least 400 g/L. Each possibility may be considered as being a separate embodiment of the present invention.

### Cellulolytic Bacteria

As mentioned, methods and compositions of the present invention utilize a cellulolytic bacterium. The terms “cellulolytic bacterium,” “cellulose-hydrolyzing bacterium,” and “cellulosic bacterium” as used herein are synonymous and refer to a bacterium capable of hydrolyzing cellulosic biomass into soluble sugars that support bacterial proliferation. The hydrolysis can be either in nature or in an artificial system such as a bioreactor. Exemplary, non-limiting cellulolytic bacterium examples of cellulolytic bacterium are Clostridium thermocellum (American Type Culture Collection [ATCC] Number 27405), Clostridium papyrosolvens (ATCC #700395), Cellulomonas sp. (ATCC #21399), Thermobifida fusca (a.k.a. Thermomonospora fusca; ATCC #27730), Thermoanaerobacter ethanolicus (ATCC #31550), Acetivibrio cellulolyticus (ATCC #33288), Clostridium populeti (ATCC #35295), Clostridium cellulovorans (ATCC #35296), Clostridium sp. (ATCC #39045), Teredinibacter turnerae (a.k.a. Teredinobacter turnerae; ATCC #39867), Clostridium stercorarium subsp. thermolacticum a.k.a. Clostridium thermolacticum; ATCC #43738), Ruminococcus flavefaciens (ATCC #49949), Fibrobacter intestinalis (ATCC #49950), Clostridium hungatei (ATCC #700212 and 700213), Cellulomonas persica (ATCC #700642), Cellulomonas iranensis (ATCC #700643), Caldicellulosiruptor kristjanssonii (ATCC #700853), Thermobifida fusca (ATCC #BAA-629). In another embodiment, the cellulolytic bacterium utilized in methods and compositions of the present invention is selected from the above species. In another embodiment, the cellulolytic bacterium is one or more of any of the above species. In other embodiments, the cellulolytic bacterium of methods and compositions of the present invention can be any other cellulolytic bacterium known in the art. Many cellulose-degrading microbes are known in the art and can be obtained, for example from the ATCC. Each possibility may be considered as being a separate embodiment of the present invention.

In another embodiment, the cellulolytic bacterium belongs to the genus Clostridium. In certain preferred embodiments, the cellulolytic bacterium is Clostridium thermocellum. In another embodiment, the cellulolytic bacterium is C. acetobutylicum. In another embodiment, the cellulolytic bacterium is C. ljungdahlii. In another embodiment, the cellulolytic bacterium is selected from the group consisting of Clostridium thermocellum ATCC #27405, Clostridium papyrosolvens, ATCC #700395 Clostridium sp. JC3 strain (FERM P-19026), and Clostridium thermocellum ATCC #31549. In another embodiment, the cellulolytic bacterium is any other Clostridium species known in the art. Each possibility may be considered as being a separate embodiment of the present invention.

In certain embodiments, the cellulolytic bacterium of methods and compositions of the present invention is a thermophilic bacterium. The term “thermophilic bacterium” as used herein refers to a bacterium that thrives at temperatures over 45° C. In other embodiments, the term refers to a bacterium that thrives at temperatures over 55° C. In other embodiments, the term refers to a bacterium that thrives at temperatures over 65° C. In other embodiments, the term refers to a bacterium that thrives at temperatures of 45-80° C. In other embodiments, the term refers to a bacterium that thrives at temperatures of 45-90° C. In another embodiment, the thermophilic bacterium is any thermophilic bacterium known in the art. Each possibility may be considered as being a separate embodiment of the present invention.

In other embodiments of methods of the present invention, the cellulolytic bacterium is a thermophilic bacterium and the step of incubating is performed at a temperature over 40° C. In other embodiments, the temperature is over 50° C. In other embodiments, the temperature is at least 60° C. In other embodiments, the temperature is at least 65° C. In other embodiments, the temperature is at least 70° C. In other embodiments, the temperature is between 40-90° C. In other embodiments, the temperature is between 50-90° C. In other embodiments, the temperature is between 60-90° C. In other embodiments, the temperature is between 70-90° C. In other embodiments, the elevated temperature facilitates recovery of ethanol or other volatile end products. Each possibility may be considered as being a separate embodiment of the present invention.

In certain embodiments, the cellulolytic bacterium of methods and compositions of the present invention is an anaerobic bacterium. The term “anaerobic bacterium” as utilized herein refers to an organism that does not require oxygen for growth. In various embodiments, the anaerobic bacterium utilized in methods and compositions of the present invention may be inter alia an obligate anaerobe, an aerotolerant organism, or a facultative anaerobe. In another embodiment, the cellulolytic bacterium is a thermophilic anaerobic bacterium. Each possibility may be considered as being a separate embodiment of the present invention.

In another embodiment of methods of the present invention, the cellulolytic bacterium is an anaerobic bacterium and the step of incubating is performed under anaerobic conditions. In another embodiment, the incubation is performed under substantially anaerobic conditions. “Substantially anaerobic conditions,” in another embodiment, refers to conditions wherein oxygen is not detectable using standard methods; e.g. a Clark oxygen electrode. In another embodiment, the term refers to a dissolved oxygen concentration of less than 1 mg/L. In another embodiment, the term refers to a dissolved oxygen concentration of less than 0.3 mg/L. In another embodiment, the term refers to a dissolved oxygen concentration of less than 0.1 mg/L. In another embodiment, anaerobiosis confers the advantage of eliminating the costly requirement for providing adequate oxygen transfer. Each possibility may be considered as being a separate embodiment of the present invention.

In another embodiment, the microbes utilized in a composition of the present invention are of a single strain. In another embodiment, the microbes comprise a plurality of species. In another embodiment, the microbes consist of a plurality of strains. According to some embodiments, a mixed culture comprising two or more cellulolytic microbes is employed. According to some embodiments, the mixed culture comprises two or more different bacteria, such as, but not limited to the Clostridia species disclosed herein. According to some further embodiments, a mixed culture of thermophilic yeasts or fungi is employed.

In another embodiment, the cellulolytic bacterium of methods and compositions of the present invention has been expanded on an inoculation medium containing cellulose or cellulosic biomass as the major energy source, prior to its inclusion in the mixture. As provided herein, in contrast to other methods that require a cellobiose-grown inoculum, certain embodiments of methods and compositions of the present invention are able to efficiently utilize inocula expanded on cellulose or cellulosic biomass. Cellulose and cellulosic biomass are significantly less expensive than cellobiose and thus provide an advantage in this regard. The term “expanded” as used herein is interchangeable with the term “amplified,” and refers to incubation under conditions wherein the bacteria in the inoculum are able to replicate. In another embodiment, the cellulose is purified cellulose. In another embodiment, other energy sources in addition to cellulose are present in the inoculation medium. In another embodiment, cellulose and/or cellulosic biomass is the only significant energy source in the inoculation medium. Each possibility may be considered as being a separate embodiment of the present invention.

In another embodiment of methods of the present invention, the step of incubating is performed for at least 30 hr. In another embodiment, the step of incubating is performed for at least 72 hr. In another embodiment, the step of incubating is performed for 30-48 hr. In another embodiment, the step of incubating is performed for at least 72 hr at a temperature over 40° C. In another embodiment, the temperature is over 50° C. In another embodiment, the temperature is at least 60° C. In another embodiment, the elevated temperature prevents contamination. Each possibility may be considered as being a separate embodiment of the present invention.

In another embodiment of methods of the present invention, the step of incubating is performed for at least 96 hours. In another embodiment, the step of incubating is performed for at least 96 hr at a temperature over 40° C. In another embodiment, the temperature is over 50° C. In another embodiment, the temperature is at least 60° C. In another embodiment, the elevated temperature prevents contamination. Each possibility may be considered as being a separate embodiment of the present invention.

### BETA-Glucosidase Enzymes

As mentioned, methods and compositions of the present invention utilize BETA-glucosidase enzymes. The BETA-glucosidase enzyme, EC 3.2.1.21, used in the present invention may be obtained commercially or produced from a microorganism. In certain embodiments, the microorganism is a bacterium. In other embodiments, the microorganism is a fungus. In another embodiment, the BETA-glucosidase enzyme is obtained from C. thermocellum. In another embodiment, the microorganism is another microorganism known in the art. In another embodiment, the BETA-glucosidase enzyme is a recombinant BETA-glucosidase enzyme. In another embodiment, the BETA-glucosidase enzyme is purified from a natural source. Each possibility may be considered as being a separate embodiment of the present invention.

In certain embodiments, the BETA-glucosidase enzyme of methods and compositions of the present invention is a thermophilic enzyme. The term “thermophilic enzyme” as used herein refers to an enzyme that is active at temperatures over 45° C. In other embodiments, the term refers to an enzyme active at temperatures over 55° C. In other embodiments, the term refers to an enzyme active at temperatures over 65° C. In other embodiments, the term refers to an enzyme active at temperatures of 45-80° C. In other embodiments, the term refers to an enzyme active at temperatures of 45-90° C. In other embodiments, the term refers to an enzyme active at temperatures of 45-95° C. In another embodiment, the thermophilic enzyme is any thermophilic enzyme known in the art. Each possibility may be considered as being a separate embodiment of the present invention.

In another embodiment, the BETA-glucosidase enzyme is a thermostable BETA-glucosidase enzyme. “Thermostable enzyme” refers, in another embodiment, to an enzyme capable of maintaining 90% function after a 12-hour incubation at 50° C. In another embodiment, the term refers to an enzyme capable of maintaining 90% function after a 12-hour incubation at 55° C. In another embodiment, the term refers to an enzyme capable of maintaining 90% function after a 12-hour incubation at 60° C. In another embodiment, the term refers to an enzyme capable of maintaining 90% function after a 12-hour incubation at 65° C. In another embodiment, the term refers to an enzyme capable of maintaining 90% function after a 12-hour incubation at 70° C. Each possibility may be considered as being a separate embodiment of the present invention.

One exemplary, non-limiting example of a BETA-glucosidase enzyme that can be utilized in methods and compositions of the present invention is encoded by the sequence set forth in SEQ ID NO: 1. This is the C. thermocellum-derived BETA-glucosidase utilized in the Examples. The amino-acid sequence for this enzyme is set forth in SEQ ID NO: 2. Many other examples of thermostable BETA-glucosidase enzymes are known in the art.

Additional exemplary, non-limiting amino acid sequences of thermostable BETA-glucosidase enzymes are: SEQ ID NO: 3 (GenBank Accession No. YP_{—}001036646), and 5 (GenBank No. X15644), each of which is from C. thermocellum; SEQ ID NO: 7 (from L. casei; GenBank No. YP_{—}001986747); SEQ ID NO: 8 (from B. thetaiotaomicron; GenBank No. NP_{—}812226); SEQ ID NO: 9 (from methanogenic Archaeon; GenBank No. YP_{—}684568); and SEQ ID NO: 10 (from D. thermophilum; GenBank No. YP_{—}002251757).

Additional exemplary, non-limiting nucleotide sequences encoding thermostable BETA-glucosidase enzymes are SEQ ID NO: 2 and SEQ ID NO: 4 (GenBank Accession No. NC_{—}009012), each of which is from C. thermocellum; and SEQ ID NO: 6 (GenBank Accession No. X15644).

The present invention is not limited to the use of thermostable BETA-glucosidase enzymes. Many other BETA-glucosidase enzyme sequences are known in the art, for example the sequence set forth in SEQ ID NO: 11 (from S. coelicolor; GenBank Accession No. NP_{—}626770); SEQ ID NO: 12 (from the fungus A. niger; GenBank No. XP_{—}001398816); SEQ ID NO: 13 (from L. monocytogenes; GenBank No. YP_{—}014348); SEQ ID NO: 14 (from S. cellulosum; GenBank No. YP_{—}001619209); SEQ ID NO: 15 (from X. campestris; GenBank No. YP_{—}001905404); SEQ ID NO: 16 (from P. atrosepticum; GenBank No. YP_{—}050881). SEQ ID NO: 17 (from L. lactis; GenBank No. YP_{—}001032747); and SEQ ID NO: 18 (from the fungus A. fumigatus; GenBank No. XP_{—}753926);

In another embodiment, the BETA-glucosidase enzyme is at least 80% homologous to a sequence selected from SEQ ID NO: 2, 3, 5, and 7-18. In another embodiment, the BETA-glucosidase is at least 85% homologous to a sequence selected from SEQ ID NO: 2, 3, 5, and 7-18. In another embodiment, the BETA-glucosidase is at least 88% homologous to a sequence selected from SEQ ID NO: 2, 3, 5, and 7-18. In another embodiment, the BETA-glucosidase is at least 90% homologous to a sequence selected from SEQ ID NO: 2, 3, 5, and 7-18. In another embodiment, the BETA-glucosidase is at least 92% homologous to a sequence selected from SEQ ID NO: 2, 3, 5, and 7-18. In another embodiment, the BETA-glucosidase is at least 94% homologous to a sequence selected from SEQ ID NO: 2, 3, 5, and 7-18. In another embodiment, the BETA-glucosidase is at least 96% homologous to a sequence selected from SEQ ID NO: 2, 3, 5, and 7-18. In another embodiment, the BETA-glucosidase is at least 98% homologous to a sequence selected from SEQ ID NO: 2, 3, 5, and 7-18. In another embodiment, the BETA-glucosidase is at least 99% homologous to a sequence selected from SEQ ID NO: 2, 3, 5, and 7-18. In another embodiment, the BETA-glucosidase has one of the above percentages of homology to a sequence selected from SEQ ID NO: 2, 3, 5, 7-11, and 13-17 (the bacterial BETA-glucosidase enzymes disclosed herein; excluding SEQ ID NO: 12 and 18, which are fungal). In another embodiment, the BETA-glucosidase has one of the above percentages of homology to a sequence selected from SEQ ID NO: 2, 3, 5, and 7-10 (the thermostable BETA-glucosidase enzymes disclosed herein). In another embodiment, the BETA-glucosidase is a variant of a sequence selected from SEQ ID NO: 2, 3, 5, and 7-18. In another embodiment, the BETA-glucosidase is a variant of a sequence selected from SEQ ID NO: 2, 3, 5, 7-11, and 13-17. In another embodiment, the BETA-glucosidase is a variant of a sequence selected from SEQ ID NO: 2, 3, 5, and 7-10. In another embodiment, the BETA-glucosidase variant or homologue exhibits BETA-glucosidase enzymatic activity. Each possibility may be considered as being a separate embodiment of the present invention.

In another embodiment, the BETA-glucosidase enzyme is at least 80% homologous to a protein encoded by a sequence selected from SEQ ID NO: 1, 4, and 6. In another embodiment, the BETA-glucosidase is at least 85% homologous to a protein encoded by a sequence selected from SEQ ID NO: 1, 4, and 6. In another embodiment, the BETA-glucosidase is at least 88% homologous to a protein encoded by a sequence selected from SEQ ID NO: 1, 4, and 6. In another embodiment, the BETA-glucosidase is at least 90% homologous to a protein encoded by a sequence selected from SEQ ID NO: 1, 4, and 6. In another embodiment, the BETA-glucosidase is at least 92% homologous to a protein encoded by a sequence selected from SEQ ID NO: 1, 4, and 6. In another embodiment, the BETA-glucosidase is at least 94% homologous to a protein encoded by a sequence selected from SEQ ID NO: 1, 4, and 6. In another embodiment, the BETA-glucosidase is at least 96% homologous to a protein encoded by a sequence selected from SEQ ID NO: 1, 4, and 6. In another embodiment, the BETA-glucosidase is at least 98% homologous to a protein encoded by a sequence selected from SEQ ID NO: 1, 4, and 6. In another embodiment, the BETA-glucosidase is at least 99% homologous to a protein encoded by a sequence selected from SEQ ID NO: 1, 4, and 6. In another embodiment, the BETA-glucosidase is a variant of a protein encoded by a sequence selected from SEQ ID NO: 1, 4, and 6. In another embodiment, the BETA-glucosidase variant or homologue retains its enzymatic activity. Each possibility may be considered as being a separate embodiment of the present invention.

The term “variant”, as used herein in the context of proteins, refers to a protein that possesses at least one modification compared to the original protein. Preferably, the variant is generated by modifying the nucleotide sequence encoding the original protein and then expressing the modified protein using methods known in the art. A modification may include at least one of the following: deletion of one or more nucleotides from the sequence of one polynucleotide compared to the sequence of a related polynucleotide, the addition of one or more nucleotides or the substitution of one nucleotide for another. Accordingly, the resulting modified protein may include at least one of the following modifications: one or more of the amino acid residues of the original protein are replaced by different amino acid residues, or are deleted, or one or more amino acid residues are added to the original protein. Other modification may be also introduced, for example, a peptide bond modification, cyclization of the structure of the original protein. A variant may have an altered binding ability to a cellulase substrate than the original protein, altered stability at 60° C., altered specific activity or altered binding capacity to cellulosome, etc. A variant may have at least 50% identity with the original cellulose binding region, preferably at least 60% or at least 70% identity.

In another embodiment, a BETA-glucosidase enzyme utilized in the present invention further comprises a cellulose-binding domain (CBD). In another embodiment, a BETA-glucosidase enzyme further comprises an affinity tag for selection and isolation of the protein product encoded by same. Examples of such an affinity tag include, but are not limited to, a polyhistidine tract, polyarginine, glutathione-S-transferase (GST), maltose binding protein (MBP), a portion of staphylococcal protein A (SPA), and various immunoaffinity tags (e.g. protein A) and epitope tags such as those recognized by the EE (Glu-Glu) antipeptide antibodies. The affinity tag may also be a signal peptide either native or heterologous to baculovirus, such as honeybee mellitin signal peptide. The affinity tag may be positioned at either the amino- or carboxy-terminus of the donor DNA. The constructs may also include at least one polynucleotide encoding an antibiotic resistant gene, as a selection marker.

Bacteria, fungi, and yeast that produce BETA-glucosidase are available commercially inter alia from culture collections such as the ATTC. Exemplary, non-limiting examples of bacteria that produce BETA-glucosidase are Bacillus coagulans (ATCC #7050), Bacillus cereus (ATCC #7064), Lactobacillus rhamnosus (ATCC #7469), Klebsiella pneumoniae subsp. rhinoscleromatis (ATCC #13884), Klebsiella pneumoniae subsp. ozaenae (ATCC #13885), Klebsiella pneumoniae subsp. pneumoniae (ATCC #13886, 15574, and 23357), Agrobacterium sp. (ATCC #21400), Bacillus sp. (ATCC #31068, 31069, 31070, 31071, 31072, 31073, 31074, 31075, 31076, and 31077), and Enterobacter cloacae subsp. cloacae (ATCC #39978). In another embodiment, the bacterium utilized to produce recombinant BETA-glucosidase is selected from the above species. In another embodiment, the bacterium is one or more of any of the above species. Each possibility may be considered as being a separate embodiment of the present invention.

Exemplary, non-limiting examples of fungi and yeast that produce BETA-glucosidase are Candida molischiana (ATCC Number 2516), Aspergillus niger (ATCC #6275, 16888, and 66371), Penicillium ochro-chloron (ATCC #9112), Candida albicans (ATCC #10261, 38247, and 64385), Eupenicillium brefeldianum (ATCC #10417), Eupenicillium parvum (ATCC #10479), Trichoderma reesei (ATCC #13631), Aspergillus quadricinctus (ATCC #16897), Candida cacaoi (ATCC #18736), Septoria lycopersici (ATCC #18835), Aspergillus oryzae (ATCC #20423), Cryptococcus curvatus (ATCC #20509), Aureobasidium sp. (ATCC #20524), Phanerochaete chrysosporium (ATCC #20696), Phoma strasseri (ATCC #24146), Trichoderma reesei (ATCC #24449), Cryptococcus tsukubaensis (ATCC #24555), Disporotrichum dimorphosporum (ATCC #24562), Aspergillus nidulans (ATCC #24704), Sporotrichum pruinosum (ATCC #24782), Penicillium melinii (ATCC #24783), Penicillium oxalicum (ATCC #24784), Diplodia gossypina (ATCC #26123), (ATCC #26501) (undescribed basidiomycete), Saccharomyces cerevisiae (ATCC #26786 and 90918), Thermoascus aurantiacus (ATCC #26904), Trichoderma reesei (ATCC #26921), Phanerochaete chrysosporium (ATCC #32629), Thermoascus aurantiacus (ATCC #34115), Candida wickerhamii (ATCC #36540), Neurospora sitophila (ATCC #36935), Schizophyllum commune (ATCC #38548), Stemphylium loti (ATCC #38587), Pisolithus tinctorius (ATCC #42409), Sporotrichum thermophile (ATCC #42464), Scopulariopsis sp. (ATCC #44206), Penicillium janthinellum (ATCC #44750), Sclerotium glucanicum (ATCC #46347), Trichoderma reesei (ATCC #46480), Trichoderma reesei (ATCC #46481), Thermoascus aurantiacus (ATCC #46993), Myceliophthora thermophile (ATCC #48104), Geotrichum candidum (ATCC #48590), Aspergillus phoenicis (ATCC #52007), Aspergillus terreus (ATCC #52293), Trichoderma harzianum (ATCC #52324), Trichoderma longibrachiatum (ATCC #52326), Aspergillus terreus (ATCC #52430), Dekkera bruxellensis (ATCC #52904), Trichoderma longibrachiatum (ATCC #60641), Humicola sp. (ATCC #60849), Aspergillus terreus (ATCC #64107), Penicillium oxalicum (ATCC #64198), Thermoascus aurantiacus (ATCC #64510), Mucor hiemalis (ATCC #66567), Trichoderma atroviride (ATCC #74058), Thermomyces lanuginosus (ATCC #76323), Cochliobolus carbonum (ATCC #90305), Botryotinia fuckeliana (ATCC #90479), Botryotinia fuckeliana (ATCC #90480), Penicillium pinophilum (ATCC #200400, 200401, 200402, 200403, and 200404), Trichoderma harzianum (ATCC #201359), and Trichoderma harzianum (ATCC #20476). In another embodiment, the fungus or yeast utilized to produce recombinant BETA-glucosidase is selected from the above species. In another embodiment, the fungus or yeast is one or more of any of the above species. Each possibility may be considered as being a separate embodiment of the present invention.

In certain embodiments, the BETA-glucosidase enzyme utilized in methods and compositions of the present invention may be capable of hydrolyzing both cellobiose and higher cellodextrins. In another embodiment, the BETA-glucosidase enzyme is a recombinant BETA-glucosidase enzyme. In another embodiment, the BETA-glucosidase enzyme is purified from a natural source. In another embodiment, an additional enzyme is added to a mixture or cellulose hydrolysis apparatus of the present invention. In another embodiment, more than one additional enzyme is added. In another embodiment, the BETA-glucosidase enzyme is the only recombinant enzyme added. In another embodiment, non-purified or partially purified enzyme is added. Each possibility may be considered as being a separate embodiment of the present invention.

In another embodiment of the present invention, another enzyme capable of hydrolyzing a larger cellodextrin is utilized in addition to the BETA-glucosidase enzyme present in methods and compositions of the present invention. In another embodiment, an enzyme having activity for cellotriose or cellotetraose is utilized. Each possibility may be considered as being a separate embodiment of the present invention.

### Advantageous Features

In certain embodiments, methods and compositions of the present invention are capable of hydrolyzing cellulose at a rate significantly greater than comparable methods lacking one or more features of the present invention. In another embodiment, a method of the present invention hydrolyzes cellulose at a rate at least 20% higher than a comparable method in the absence of exogenous BETA-glucosidase enzyme. In another embodiment, the cellulose hydrolysis rate is at least 20% higher than conditions wherein the pH is maintained at a value consistent with bacterial replication and/or metabolism for the entire hydrolysis reaction. In another embodiment, the rate is at least 20% higher than conditions wherein the pH is uncontrolled for the entire hydrolysis reaction. In another embodiment, the rate enhancement is at least 30%. In another embodiment, the rate enhancement is at least 50%. In another embodiment, the rate enhancement is at least 70%. In another embodiment, the rate enhancement is at least 100%. In another embodiment, the rate enhancement is at least 150%. Each possibility may be considered as being a separate embodiment of the present invention.

In certain embodiments, the yield of reducing sugar is significantly more than that obtainable by comparable methods and compositions lacking one or more features of the present invention. In another embodiment, a method of the present invention produces at least 30% more reducing sugars than a comparable method in the absence of exogenous BETA-glucosidase glucosidase enzyme. In another embodiment, the yield is at least 30% higher than that obtainable in conditions wherein the pH is maintained at a value consistent with bacterial replication for the entire fermentation reaction. In another embodiment, the yield is at least 30% higher than that obtainable in conditions wherein the pH is uncontrolled for the entire fermentation reaction. In another embodiment, the yield enhancement is at least 50%. In another embodiment, the rate enhancement is at least 70%. In another embodiment, the yield enhancement is at least 100%. In another embodiment, the yield enhancement is at least 150%. In another embodiment, at least 15 g/L glucose is produced by the end of the fermentation. In another embodiment, at least 20 g/L glucose is produced. In another embodiment, at least 25 g/L glucose is produced. In another embodiment, at least 30 g/L glucose is produced. Each possibility may be considered as being a separate embodiment of the present invention.

### EXPERIMENTAL DETAILS SECTION

### MATERIALS AND EXPERIMENTAL METHODS (EXAMPLES 1-7)

### Chemicals and Enzymes

Microcrystalline cellulose was obtained from Merck KGaA, 64271 Darmstadt, Germany. BETA-glucosidase (Novozyme 188, from Novozyme A/S, Krogshoejvej 36 2880, Bagsvaerd Denmark) was obtained from the local agent of Novo Industries a/s.

All chemicals were purchased from Sigma-Aldrich (St. Louis, Mo., USA) unless otherwise noted.

### Microorganisms

Clostridium thermocellum was kindly provided by Raphael Lamed (Tel Aviv University, Israel) and is available from the ATCC (American Type Culture Collection, Manassas, Va. 20108, USA, catalogue #27405).

Stock culture was maintained in a CT medium (see below) with addition of glycerol to a final concentration of 25% (v/v). Stocks were stored at −80° C.

Batch cultures on microcrystalline cellulose (Avicel™) or cellobiose were grown at 60° C. in sealable serum vials (Wheaton Science Products, Millville, N.J. 08332-2038, USA) containing 15 ml of broth in 25 ml volume vial.

### Medium Composition and Preparation

Each liter of the CT medium used in the batch experiments contained the following: 0.5 gr KH₂PO₄, 0.5 g K₂HPO₄, 0.5 gr MgCl₂*6H₂O, 10.5 gr MOPS (moropholinopropa acid), 1.3 gr (NH₄)₂SO₄, 5 gr Yeast Extract (Laboratorios Conda C, La Forja, 9 28850 Torrejón de Ardoz•Madrid), 1 mg Resazurin, 1 gr Cysteine HCL and 8 gr cellobiose or cellulose as indicated in the figures. The pH of the medium was adjusted to pH 7.2 using 1 M NaOH solution. The medium was prepared in aluminum crimp-seal serum vials under N₂ gas prior to autoclaving.

### Purification of Enzymes

### Purification of Novozyme 188 (A. Niger BETA-Glucosidase)

An aliquot of 20 ml of crude BETA-glucosidase from Novozymes (Novozym 188, Novozymes A/S, Krogshoejvej 36 2880 Bagsvaerd Denmark) was dialyzed twice in 4° C. against 4 liter of 10 mM phosphate buffer pH 6.4 (phosphate buffer). DEAE-cellulose (Amersham Biosciences AB SE-751 84 Uppsala, Sweden) was washed with phosphate buffer and packed in a column in a final volume of 20 ml. The column was then washed with 100 ml of phosphate buffer. The dialyzed crude enzyme sample was then loaded on the column at a linear flow rate of 2 ml/min. After loading, the column was washed again with phosphate buffer until the OD₂₈₀ reached the value of 0.1. A linear gradient of increasing NaCl concentration was applied, and fractions of 2 ml were collected at the same flow rate. The peak of BETA-glucosidase was determined by the PNPG assay. The soluble fractions were analyzed by SDS-PAGE. The proper fractions (purity>80%) were pooled, diluted by glycerol (50% V/V), divided into small aliquots, and stored at −20° C.

Purification of BETA-Glucosidase from Clostridium Thermocellum

The nucleotide sequence of the Clostridium thermocellum-derived BETA-glucosidase is set forth in SEQ ID NO: 1. The BETA-glucosidase coding sequence is identical to GenBank #X60268, except for sequence encoding the second residue, changed from serine to alanine for cloning reasons, and the histidine tag added for purification reasons. The corresponding amino-acid sequence is set forth in SEQ ID NO: 2.

Bacterial cells (500 ml of induced culture) from BL21 (DE3) carrying plasmid pET-28a-BGLA-Ct and induced for 5 hours (hr) at 37° C. with 0.1 mM IPTG were centrifuged, and the resulting pellet was suspended in 10 ml TBS and disrupted by sonication, 80% amplitude, 5 cycles of 2 min each). The total cell extract was centrifuged at 15,000 g for 15 min at 4° C., and the soluble fraction was collected. The supernatant was then affinity purified on an Ni-IDA column (Amersham Biosciences AB SE-751 84 Uppsala, Sweden) that was equilibrated with TBS as a binding buffer. The recombinant His-Tag-BETA-Glucosidase was eluted from the column using linear gradient (10-500 mM imidazole in TBS with no additional supplementation using an AKTA-prime system FPLC (Amersham Biosciences AB SE-751 84 Uppsala, Sweden). The peak of BETA-glucosidase was determined by the PNPG assay. Soluble fractions were analyzed by SDS-PAGE, followed by Coomassie brilliant blue staining The proper fractions (purity>90%) were pooled. The amount of protein was determined optically by reading optical density at 280 nm. The pooled fraction was diluted by glycerol (50% VAT), divided into small aliquots, and stored at −20° C.

### Enzymatic Activity and Analysis

### PNPG Assay

The PNPG assay was used to determine the presence of BETA-glucosidase during the purification procedure for this enzyme. In addition, this procedure was used to determine the specific activity of the enzymes before the addition of this enzyme into the bacterial medium. For this purpose, a PNPG stock solution (40 mM in DDW) and 0.1 M citrate buffer pH=6 were prepared. The sample to be measured was applied into an Eppendorf tube (5 μl), and 100 μl of PNPG stock solution and 95 μl citrate buffer were added into the same tube to a total volume of 200 μl. The tube was incubated at 50° C. or 60° C. (BETA-glucosidase from Novozyme or C. thermocellum, respectively). A 200 μl sample was drawn after the completing the assay and transferred into a 96-well plate. Absorbance at 412 nm was measured using an ELISA reader.

### TLC Analysis

Substrate breakdown from hydrolysis of cellulose by C. thermocellum was analyzed by TLC. Aliquots (1 μl) were applied to TLC plates, which were eluted with n-butanol, ethyl acetate, 2-propanol, acetic acid and water (1:3:2:1:1), then visualized by heating after spraying with a 1:1 (V/V) mixture of 0.2% menthanolic orcinol and 20% sulfuric acid.

### Residual Cellulose Quantification

The amount of cellulose during the accelerated bacterial hydrolysis by Clostridium thermocellum was monitored by weighing the cellulose present. Serum flasks were thoroughly mixed, and 1-2 ml of growth medium was withdrawn using a sterile syringe and transferred into pre-weighed 2 ml Eppendorf tubes. Samples were centrifuged to remove the liquid and then washed 2 times with double distilled water to remove residual salts. Tubes were dried at 60° C. for 48 hr. Weights of tubes with dried cellulose samples were determined, and the residual amount of cellulose was calculated by subtracting the weight of the empty tubes.

### Reducing Sugar Quantification

The quantity of reducing sugars produced was estimated calorimetrically using dinitrosalycylic acid reagent (DNS reagent). 100-μl aliquots of serially diluted samples were added into Eppendorf tubes, followed by 150 μl DNS reagent. Glucose was used for standard curve. Eppendorf tubes were thoroughly mixed, centrifuged for 10 seconds, and incubated in 100° C. water bath for 5 min. A sample of 200 μl was drawn from the tube and transferred into a 96-well plate, and absorbance was measured at 540 nm was using an ELISA reader.

### Quantification of Substrate and Batch Culture Fermentation Products

Samples were withdrawn during the batch fermentation using a sterile syringe (18 Gauge) and, if not immediately processed, were frozen at −20° C. The flask was thoroughly mixed by vortexing and a 1-2 ml sample was drawn into a clean 2 ml Eppendorf tube. Amounts of reducing sugar produced were estimated calorimetrically using DNS reagent as described above.

### Example 1

### Cloning, Expression and Purification of a Recombinant C. Thermocellum BETA-Glucosidase (B21A) Protein

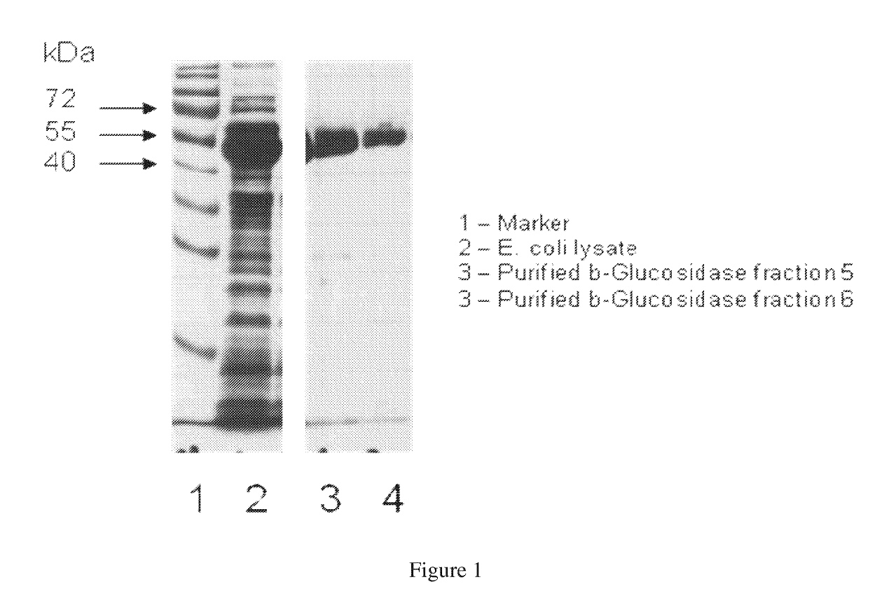

A plasmid containing the gene encoding the BETA-glucosidase of the Clostridium thermocellum cellulosome (SEQ ID NO: 1; FIG. 1) was used to transform E. coli strain BL21 (DE3; Novagen, WI, USA). Transformed cells were grown on LB medium with appropriate antibiotics and IPTG (for induction) for 3-5 hr at 37° C. The cell culture was centrifuged, resuspended in Tris buffer (50 mM, pH 7.2), sonicated, and re-centrifuged. BETA-glucosidase was purified as described in the Methods section, yielding highly purified protein (FIG. 1, lanes 1-4). The molecular weight of the purified product was in agreement with the theoretical calculated value.

### Example 2

### Addition of External BETA-Glucosidase Enhances Hydrolysis of Microcrystalline Cellulose by C. Thermocellum

The effect of adding exogenous C. thermocellum BETA-glucosidase on the bacterial hydrolysis of microcrystalline cellulose (MC) by C. thermocellum was evaluated using two different amounts of BETA-glucosidase, under standard growth conditions, i.e., MC at 21 gr per liter of growth medium (g/L). 25 ml serum flasks with 15 ml growth medium and 2.1% MC w/v were inoculated with 1 ml C. thermocellum inoculum that had been grown on cellobiose, and the flasks were allowed to acclimatize for 1 hr under continuous agitation at 60° C. 0.3 or 0.6 ml of recombinant C. thermocellum BETA-glucosidase or PBS (negative control) was added into the flask. Flasks were mixed, and a 3-ml. sample from each flask was withdrawn using a sterile syringe. Flasks were allowed to incubate under the same conditions and were sampled every 12 hr. Withdrawn samples were washed, and residual cellulose was measured as described in the Methods section.

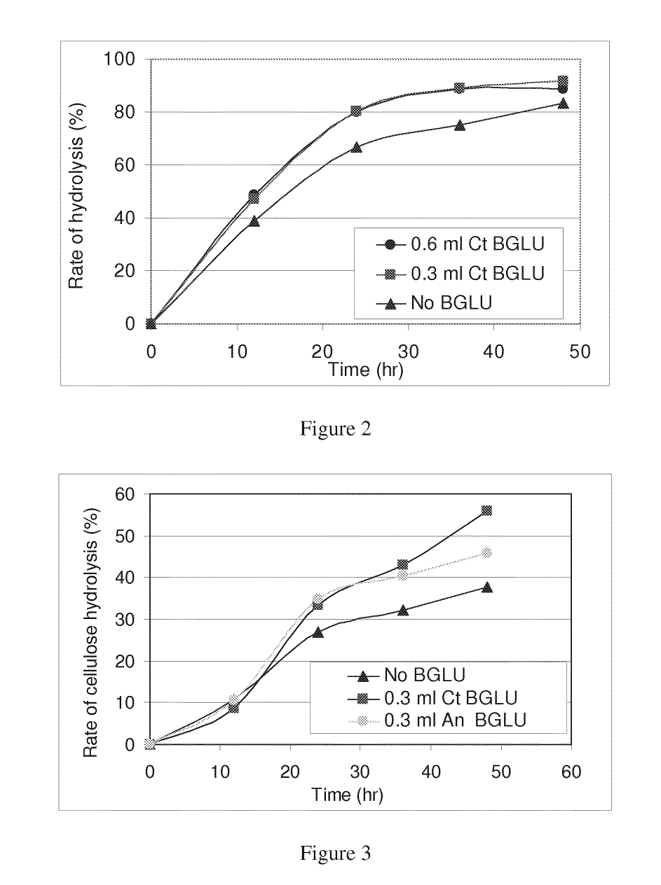

Addition of either amount of BETA-glucosidase to the growth medium increased the level of cellulose solubilization (FIG. 2) by 10% of the total amount of cellulose, namely 49% vs. 39%, at 12 hours post-inoculation and 13-15% of total cellulose at the 24- and 36-hour timepoints. About 90% solubilization was observed for the BETA-glucosidase-containing samples after 48 hours, vs. 83% solubilization for C. thermocellum alone.

In addition and also unexpectedly, inclusion of BETA-glucosidase caused stimulation of bacterial growth on the insoluble substrate, as indicated by a change in the color of the substrate during the hydrolysis process from white (native color) to pale-yellow, then deep-yellow late in the hydrolysis process. The color change was associated with colonization of the bacteria on the cellulose and was particularly evident 12-24 hours after inoculation.

### Example 3

### The Effect of the Source of the BETA-Glucosidase and the Initial Amount of Substrate on Hydrolysis of Microcrystalline Cellulose by C. Thermocellum

The next experiment compared the effect of C. thermocellum BETA-glucosidase vs. commercial purified BETA-glucosidase from A. niger (Novozymes) on the hydrolysis rate of MC by C. thermocellum. In this and subsequently reported experiments, a concentration of 21 g/L of cellulose was utilized, except where otherwise indicated. The activities of the A. niger and C. thermocellum enzymes were compared using the chromomeric substrate PNPG, the results of which were used to normalize the amounts added to the growth medium in order that equal specific activities were added. Since the specific activities of the two enzymes were found to be the same, equal amounts of the two enzymes were added. 25 ml flasks containing 15 ml growth medium were inoculated with 1 ml C. thermocellum and allowed to acclimatize for 1 hr under continuous agitation at 60° C., after which an aliquot of 0.3 ml of A. niger, C. thermocellum BETA-glucosidase, or PBS (control) was added, followed by analysis as described for Example 2. In addition to residual cellulose, amounts of reducing sugar were also measured.

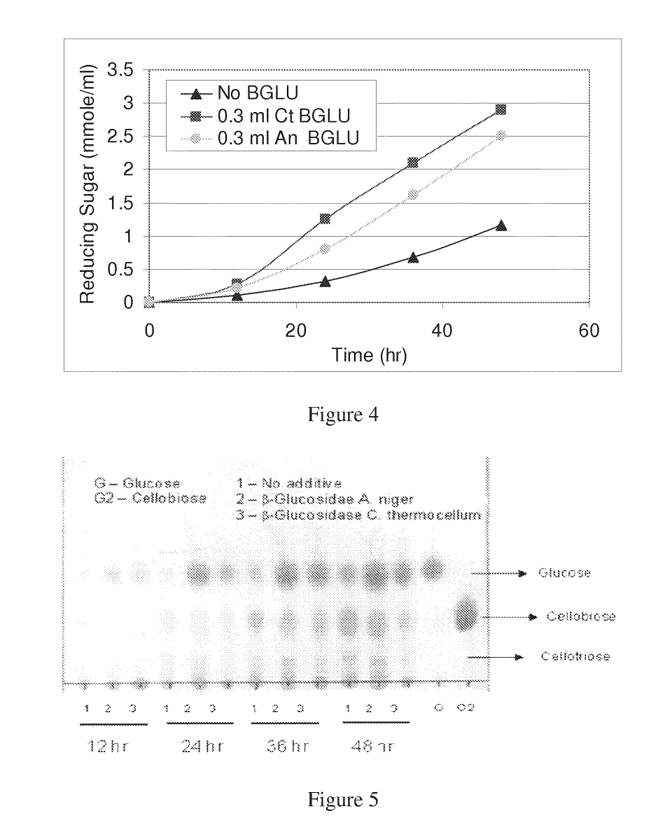

BETA-glucosidase from both sources clearly accelerated hydrolysis of MC, with C. thermocellum BETA-glucosidase being the more potent of the two enzymes, as shown by measurement of residual cellulose. At the 48-hr time point, 37% hydrolysis of total cellulose was observed with bacteria alone. Hydrolysis in the presence of the C. thermocellum and A. niger enzymes was enhanced by a difference of 18% and 12% of total cellulose, respectively, corresponding to almost a one-fold enhancement with the C. thermocellum enzyme (FIG. 3). At 24 and 36 hours, the enhancement was 10% of total cellulose for both enzymes. Measurement of soluble reducing sugar (FIG. 4) yielded much higher estimation of enhancement and confirmed the conclusions from measurements of residual cellulose. In this case, 3-4 times more reducing sugar was observed for samples containing external BETA-glucosidase. Since the number of reducing sugars of one mole of glucose is twice than of cellobiose, this corresponds to a 1.5-2-fold enhancement for addition of C. thermocellum BETA-glucosidase and a somewhat lower enhancement for the A. niger enzyme. Without wishing to be bound by theory, it appears under the conditions utilized that the superior activity of the C. thermocellum enzyme may be due to its superior thermo-stability relative to the A. niger enzyme.

TLC analysis of the final reaction products of bacterial hydrolysis was performed for samples withdrawn after 12, 24, 36 and 48 hr. This analysis confirmed that most of the cellobiose was converted to glucose after a 48-hr incubation with C. thermocellum BETA-glucosidase; however, a small but detectable amount of residual cellobiose remained. Longer cellodextrins (longer than 3 carbon atoms) were not present (FIG. 5). In contrast, a larger amount of unprocessed cellobiose and detectable amounts of longer cellodextrins were present after 48 hours in the samples containing A. niger BETA-glucosidase. This was also true of the samples containing C. thermocellum alone, although these samples had a significantly lower amount of glucose.

### Example 4

### External BETA-Glucosidase also Enhances Hydrolysis Under High MC Loading Conditions

In the past, C. thermocellum has been grown in medium containing 5-20 gr/L of cellulose. However, this low loading value is not ideal for industrial production for either soluble sugar production or ethanol fermentation. Ability to load higher amounts of cellulose would confer many advantages, including reducing the size of the infrastructure needed for ethanol fermentation and other industrial fermentations and the costs associated therewith; and eliminating the need to concentrate soluble sugar before chemical fermentation processes requiring a high initial concentration of soluble sugar. Accordingly, medium containing 40 or 80 g/L microcrystalline cellulose was prepared, inoculated with C. thermocellum, and incubated as described in the previous Example. In this case, however, recombinant BETA-glucosidase was added twice, once shortly after inoculation and then after 24 hr. Samples were taken at 0, 24, 48, 60, and 72 hr after inoculation.

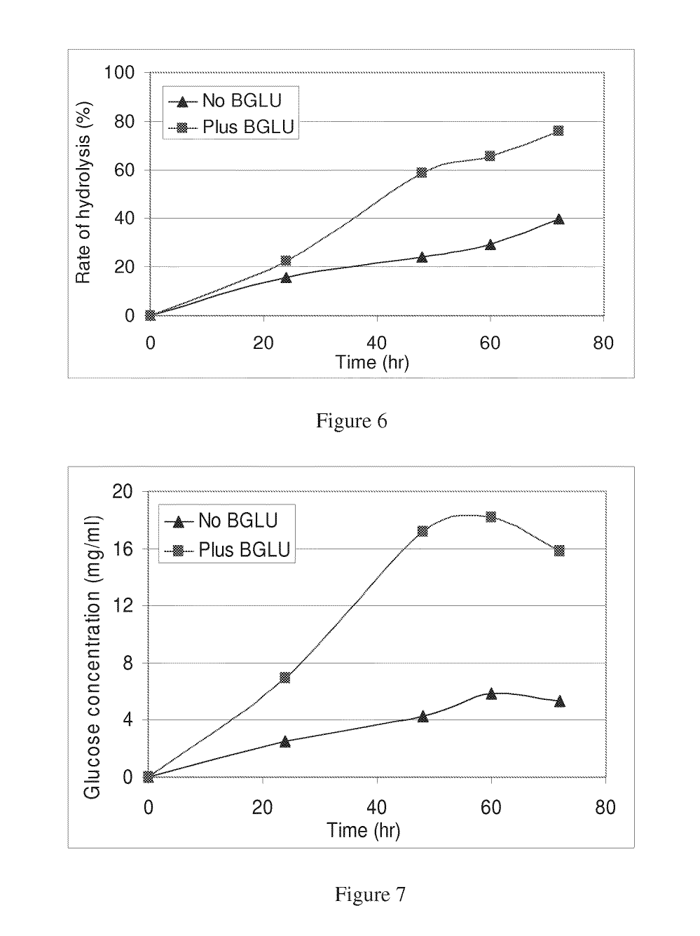

BETA-glucosidase clearly accelerated hydrolysis of 40 g/L microcrystalline cellulose at every time point tested under these conditions as well. Even at the first time point, 24 hr, BETA-glucosidase conferred a 7% increase in hydrolysis as measured by residual cellulose content (FIG. 6). After 48 hr, excellent yield and a very large enhancement were observed in the BETA-glucosidase-supplemented samples, namely close to 80%, vs. 40% for C. thermocellum alone. Enhancement by inclusion of BETA-glucosidase was also evident in measurements of soluble reducing sugar accumulation (FIG. 7); 7 mg/ml of soluble sugar was present after 24 hr in the presence of BETA-glucosidase vs. 2.5 mg/ml for the bacteria alone. At 48- and 60 hr post inoculation, about 4 times more soluble reducing sugar was present in the samples containing BETA-glucosidase.

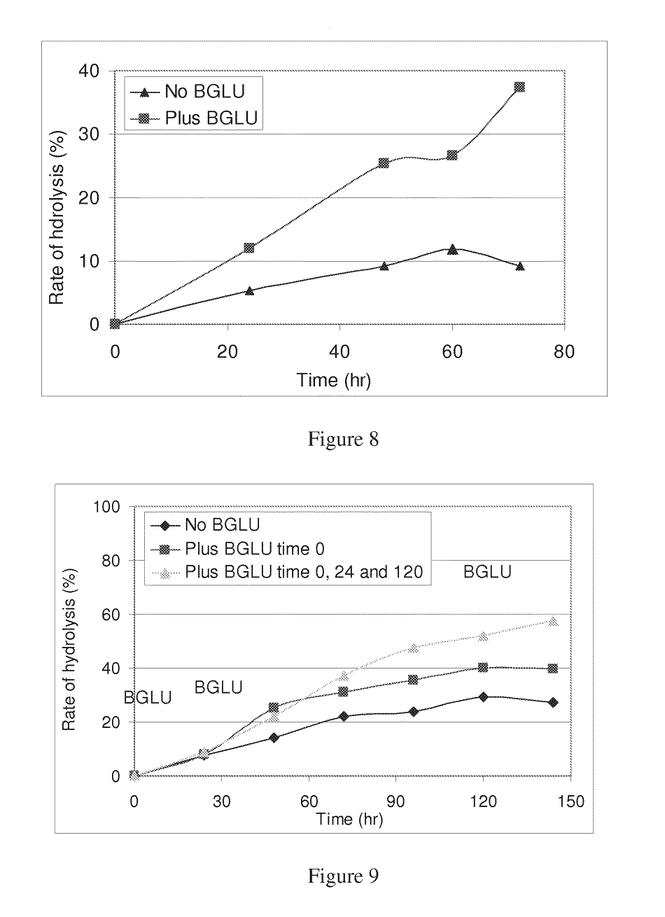

Similar results were obtained with 80 gr/L of microcrystalline cellulose (FIG. 8). In this case, the total reduction in residual cellulose after 72 hr was about 40% in the BETA-glucosidase-supplemented samples vs. 10% for C. thermocellum alone. Acceleration following addition of BETA-glucosidase was also observed at the 24-hr time point, namely 12% hydrolysis vs. 5% for bacteria alone.

### Example 5

### Sequential Addition of BETA-Glucosidase Further Enhances MC Hydrolysis Under High Loading Conditions

In an attempt to further improve hydrolysis of 80 gr/L of microcrystalline cellulose, the hydrolysis period was significantly increased to 144 hr, and BETA-glucosidase was added either not at all, at three points (shortly after the inoculation and after 24 and 120 hr), or once after the inoculation. Samples were taken at 0, 24, 48 72, 96, 120, and 144 hr after inoculation.

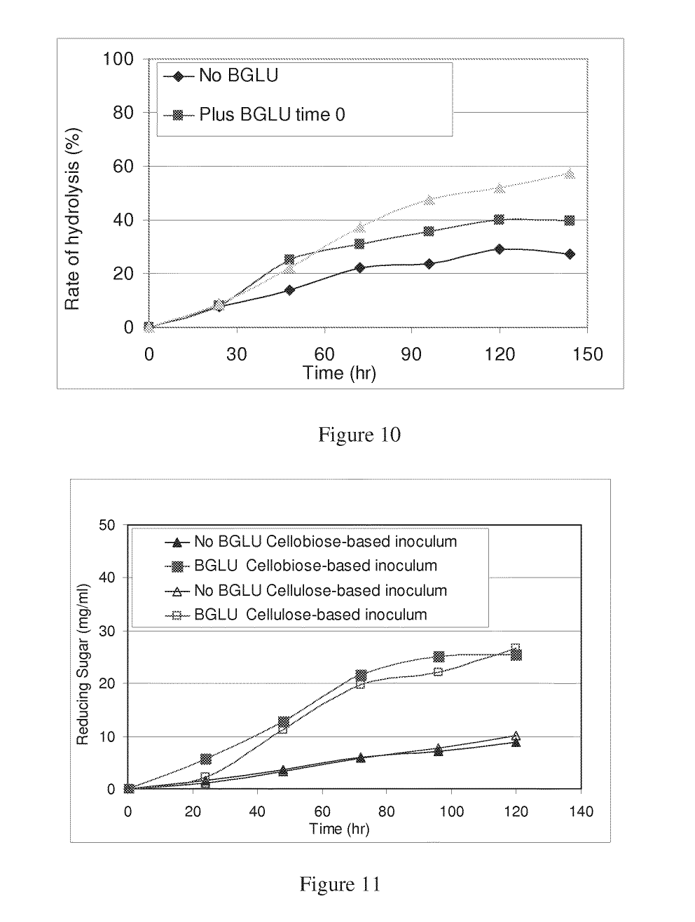

As in the previous Example, measurement of residual cellulose revealed accelerated hydrolysis in the presence of BETA-glucosidase after 48 hr (FIG. 9). In addition, repeated addition of BETA-glucosidase conferred a significant advantage vs. a single addition. This was measurable from the 72 hr time point onward. The total reduction in residual cellulose in the group receiving multiple supplementations was about 60%, vs. 40% for a single supplementation. Continued hydrolysis was observed in the multiple-supplemented group but not the other two groups, resulting in a gradually increasing margin with respect to these groups, until the last time point measured (144 hr.). These results were confirmed by measurement of reducing sugar accumulation (FIG. 10). About 35 mg of reducing sugar was accumulated for the extended administration of BETA-glucosidase, about four times the amount seen with bacteria alone. The peak of accumulation of reducing sugar with bacteria alone occurred after 120 hr for the bacteria alone and single addition samples. By contrast, in the repeated addition sample, a burst of accumulation of reducing sugar followed the third addition of BETA-glucosidase.

### Example 6

### External BETA-Glucosidase Enhances MC Hydrolysis with Both Cellobiose- and Cellulose-Grown Inocula

The previous examples utilized inoculum C. thermocellum grown on cellobiose as the sole soluble carbon source. Cellobiose is the favored carbon source for preparing C. thermocellum inoculum in small-scale experiments due to its ability to enable rapid hydrolysis of a large amount of biomass, and to reproducibly produce a relatively concentrated inoculum. However, cellobiose is a relatively expensive carbon source, disfavoring its use as a sole carbon source for large-scale commercial fermentation. For these reasons, cellulose was compared to cellobiose as a carbon source for the inoculum. C. thermocellum was grown in separate media prepared with cellobiose or cellulose. The bacteria were allowed to grow for 20 hr, and then a 1 ml aliquot was immediately used as an inoculum without further manipulations in 40 gr/liter MC-containing media. BETA-glucosidase was added at the time of inoculation and after 24 hours.

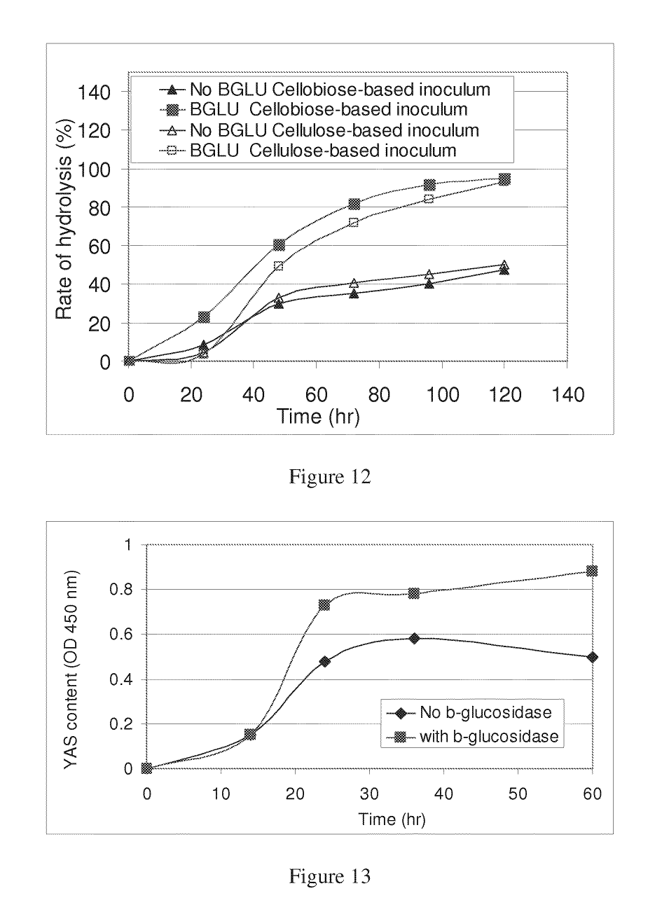

Similar results were seen with bacteria grown on cellulose vs. cellobiose, with the exception of a relatively longer lag phase in the sample inoculated with the cellulose-based inoculum and containing exogenous BETA-glucosidase, as demonstrated by measurements of both reducing sugar accumulation (FIG. 11) and residual cellulose (FIG. 12). Despite the lag, very similar values were reached with the cellulose- and cellobiose-based inocula by 96 hr, and equal or greater hydrolysis was seen with the cellulose-based inoculum by 120 hr. With both types of inocula, hydrolysis was significantly enhanced by exogenous BETA-glucosidase, even as early as the 24-hr timepoint; much larger differences were observed in at the 48 hr and subsequent timepoints.

### Example 7

### Addition of External BETA-Glucosidase Enhances Synthesis of Yellow Affinity Substance (YAS)

### Materials and Experimental Methods

### Quantification of Yellow Affinity Substance (YAS) on Microcrystalline Cellulose

Clostridium thermocellum was incubated in media containing 40 gr/L of microcrystalline cellulose as described in the above Examples. At selected time points during fermentation, a 2-ml sample was withdrawn using a syringe. The cellulose pellet was washed twice in PBS, and YAS was extracted by re-suspending the pellet of microcrystalline cellulose in 200 ml of 100% acetone, followed by incubation for 10 min at room temperature under continuous mixing and centrifugation at 14,000 rpm for 2 min. YAS was then quantified spectrophotometrically at 450 nm.

### Results

C. thermocellum produces a yellow affinity substance upon fermentation of a cellulose-containing substrate. The substance and the bacteria are firmly attached to the cellulose during the cellulose hydrolysis. Production of YAS was also observed during the fermentation of Ruminococcus flavefaciens, a cellulose-degrading bacteria in the digestive tract of ruminants. The exact chemical structure of YAS from both bacteria unknown, but it is believed to be a cartenoid-like compound. As mentioned in Example 1, inclusion of BETA-glucosidase stimulated bacterial growth on the insoluble substrate, as evidenced by a gradual change in the color of the substrate during hydrolysis from white (native color) to pale-yellow, then deep-yellow. This observation was experimentally measured in this example. Cellulose-containing growth medium was inoculated with C. thermocellum and immediately supplemented with BETA-glucosidase. Samples were withdrawn after 12, 24, 36 and 60 hours, and YAS was extracted and quantified. BETA-glucosidase supplementation increased YAS accumulation by ˜50% at the 24-hr timepoint and by a larger margin at later timepoints (FIG. 13).

### Example 8

### Addition of BETA-Glucosidase Without pH Control Enhances Hydrolysis of Pretreated Switchgrass in a 1.3-Liter Bioreactor

### MATERIALS AND EXPERIMENTAL METHODS (EXAMPLES 8-9)

### Switchgrass Pretreatment

A flask was placed in a water bath having a temperature of 20° C., and a sample of initial switchgrass was poured into the glass. Then 7-9% sodium hypochlorite containing carbonate buffer (pH=11-13) under exhaust ventilation was added at a liquid/solid ratio of 8:10 w/w. The glass was covered, and the contents were stirred for 1 hr. The sample was washed with tap, distilled and double-distilled water on a vacuum sinter-filter and then pressed until the solid content was 20-30%. The pretreated switchgrass contained 85-87% carbohydrates and 13-15% lignin and other contaminants.

### Fermentor Conditions

1.3 liter fermentor bioreactors were maintained under anaerobic conditions at 60° C., under agitation at 250 rpm. Unless indicated otherwise, the pH was kept constant at 7.2 by continuous addition of 4M NaOH solution.

### Residual Biomass Quantification

To quantify residual biomass in the bioreactor, 20 ml of solution was withdrawn from the bioreactor while under agitation using a sterile plastic pipette into 50 ml plastic tube. The tube was through mixed, and 4 4-ml samples were withdrawn into plastic tubes. The tubes were centrifuged, the liquid was removed, and tubes were dried at 60° C. for 72 hr. then weighed. Residual biomass was calculated by the weight difference relative to empty tubes.

### Results

The reaction was next scaled up to a batch culture fermentation reaction in a 1.3 L bioreactor, using 3% pretreated switchgrass biomass as the substrate. The bioreactors were inoculated with a cellobiose-grown C. thermocellum inoculum. One fermentor was supplemented with 25 mg BETA-glucosidase shortly after inoculation and at 24, 48 and 72 hr post-inoculation, while the other was not supplemented. After inoculation, the pH-controlling mechanism was switched off in order to parallel the conditions found in a serum bottle, where pH gradually decreases due to the production of acidic metabolites as a result of the fermentation process. Samples of mixed medium were withdrawn at different intervals and the amounts of soluble sugar and residual MC were determined.

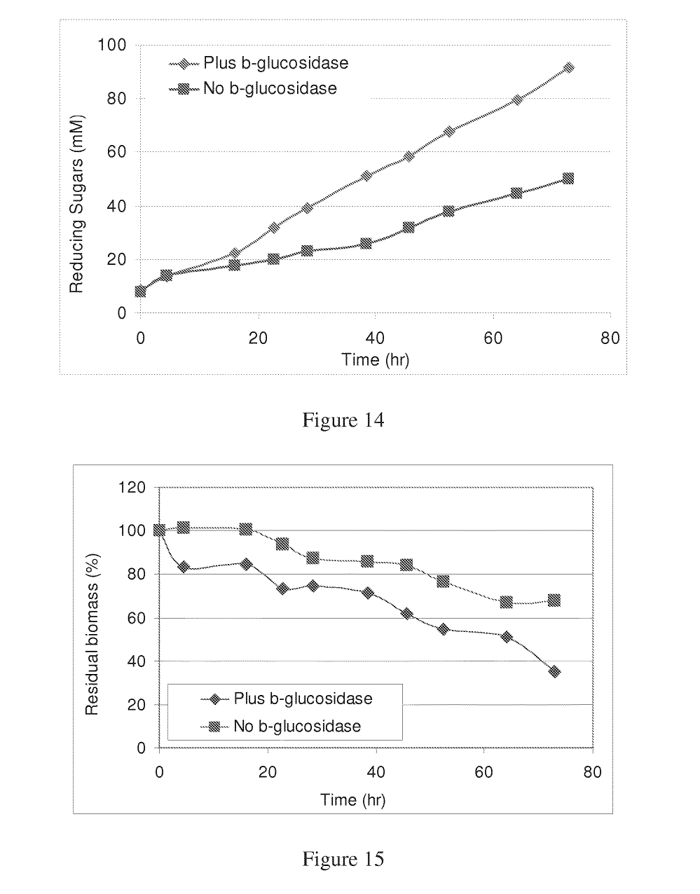

A large enhancement in soluble reducing sugar accumulation was observed in the BETA-glucosidase-supplemented fermentor (FIG. 14). In addition and in accordance with the reducing sugar data, a significant enhancement in the amount of the residual MC was observed in the BETA-glucosidase-supplemented fermentor (FIG. 15).

In conclusion, the presence of exogenous BETA-glucosidase significantly improved hydrolysis as measured by either soluble sugar accumulation or residual biomass. The advantage conferred by inclusion of exogenous BETA-glucosidase was smaller under conditions where pH was maintained (data not shown).

### Example 9

### Inclusion of Exogenous of BETA-Glucosidase Combined with “Two-Stage pH Control” Further Enhances Hydrolysis of Pretreated Switchgrass

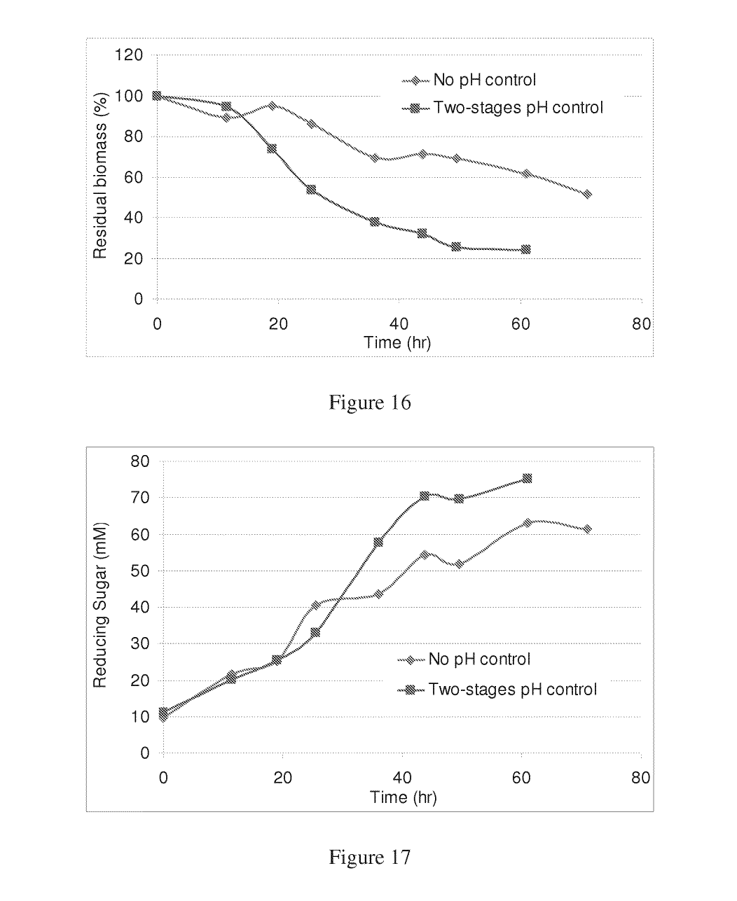

The next experiment compared a “two-stage pH control” batch culture fermentation process, wherein pH is controlled during the first part of the hydrolysis process but not the second part, with no pH control during the entire incubation. Two 1.3 liter bioreactors containing 40 gr/L of pretreated switchgrass were inoculated with a cellobiose-grown C. thermocellum inoculum; both were supplemented with 25 mg BETA-glucosidase shortly after inoculation and at 24, 48 and 72 hr post-inoculation. After inoculation, the pH-controlling mechanism in one bioreactor was switched off, while the pH in the second fermentor (the “two-stage pH control” sample) was kept at a set-point of 7.2 by dropwise addition of 4M NaOH solution. After 16 ml of NaOH had been added, which occurred between 12-24 hr, the pH-controlling mechanism in the second bioreactor was switched off, allowing the pH to decrease gradually due to the metabolic activity of the bacteria. 20 ml samples of the mixed medium were withdrawn and sampled as described for the previous Example. A large decrease in residual biomass (FIG. 16) and a significant enhancement in reducing sugar production (FIG. 17) were observed in the two-stage pH control sample

In conclusion, inclusion of exogenous BETA-glucosidase enhances hydrolysis of a variety of cellulose-containing substrates, under a variety of conditions, including both cellobiose- and cellulose-grown inocula and in both flasks and bioreactors. Sequential BETA-glucosidase addition provides still further enhancement. Inclusion of exogenous BETA-glucosidase in the absence of pH control provides a still further enhancement of batch culture fermentation of cellulose-containing substrates. The combination of exogenous BETA-glucosidase with two-stage pH control provides a still more robust enhancement.

### REFERENCES

Conchie J. 1954. 13-Glucosidase from rumen liquor. Preparation, assay and kinetics of action. Biochem. J. 58: 552-560.

Johnson, E A, E T Reese, and A L Demain. 1982. Inhibition of Clostridium thermocellum cellulase by end products of cellulolysis. J. Appl. Biochem. 4:64-71.

Kadam S K, Demain A L. 1989. Addition of cloned beta-glucosidase enhances the degradation of crystalline cellulose by the Clostridium thermocellum cellulose complex. Biochem Biophys Res Commun. 161(2):706-11

Kosugi A, Arai T, Doi R H. 2006. Degradation of cellulosome-produced cello-oligosaccharides by an extracellular non-cellulosomal beta-glucan glucohydrolase, BglA, from Clostridium cellulovorans. Biochem Biophys Res Commun. 349(1):20-3

Kotaka A et al. 2008. Direct ethanol production from barley beta-glucan by sake yeast displaying Aspergillus oryzae beta-glucosidase and endoglucanase. J Biosci Bioeng. 105(6):622-7.

Kubicek C P et al. 2009. Metabolic engineering strategies for the improvement of cellulase production by Hypocrea jecorina. Biotechnol Biofuels. 2:19.

Lamed R, Zeikus J G. 1980. Ethanol production by thermophilic bacteria: relationship between fermentation product yields of and catabolic enzyme activities in Clostridium thermocellum and Thermoanaerobium brockii. J Bacteriol 144:569-78.

Lynd L R et al. 1989. Fermentation of Cellulosic Substrates in Batch and Continuous Culture by Clostridium thermocellum. Appl Environ Microbiol. 55:3131-3139.

Ni J et al. 2007. Random exchanges of non-conserved amino acid residues among four parental termite cellulases by family shuffling improved thermostability. Protein Eng Des Sel. 20(11):535-42.

Zhang Y H et al. 2006 Outlook for cellulase improvement: screening and selection strategies. Biotechnol Adv. 24(5):452-81.

1-27. (canceled)

28. A mixture comprising a cellulolytic bacterium, a medium comprising a cellulosic feedstock, and an exogenous β-glucosidase enzyme.

29. The mixture of claim 28, wherein said exogenous β-glucosidase enzyme is an isolated β-glucosidase enzyme provided independently of said cellulolytic bacterium.

30. The mixture of claim 28, wherein said exogenous β-glucosidase enzyme is a secreted β-glucosidase enzyme that is produced by said cellulolytic bacterium.

31. The mixture of claim 28, wherein said cellulolytic bacterium is a thermophilic, anaerobic bacterium.

32. The mixture of claim 28, wherein said cellulolytic bacterium is Clostridium thermocellum.

33. The mixture of claim 28, wherein said exogenous β-glucosidase enzyme is a thermostable β-glucosidase enzyme.

34. The mixture of claim 28, wherein the cellulose concentration of said medium is at least 80 grams per liter of medium, and the pH of said medium is below 5.0.

35. A method of hydrolyzing a cellulosic feedstock, said method comprising the step of incubating, in a cellulose hydrolysis apparatus, a medium comprising said cellulosic feedstock with a cellulolytic bacterium in the presence of an exogenous β-glucosidase enzyme, thereby hydrolyzing a cellulosic feedstock.

36. The method of claim 35, wherein said exogenous β-glucosidase enzyme is an isolated β-glucosidase enzyme provided independently of said cellulolytic bacterium.

37. The method of claim 35, wherein said exogenous β-glucosidase enzyme is a secreted β-glucosidase enzyme that is produced by said cellulolytic bacterium.

38. The method of claim 35, wherein said exogenous β-glucosidase enzyme is a thermostable β-glucosidase enzyme, and said exogenous β-glucosidase enzyme is added at more than one time point to said growth media.

39. The method of claim 35, wherein said cellulolytic bacterium is a thermophilic bacterium, said enzyme is thermophilic, and the step of incubating is performed at a temperature over 40° C.

40. The method of claim 35, wherein said cellulolytic bacterium is an anaerobic bacterium, and the step of incubating is performed under substantially anaerobic conditions.

41. The method of claim 35, wherein said cellulolytic bacterium is C. thermocellum.

42. The method of claim 35, wherein said step of incubating is performed in two stages, wherein:
a. the pH of said medium is maintained at a value consistent with bacterial replication and/or metabolism during the first stage; and
b. the pH of said medium is not maintained at said level during the second stage.

43. A method of hydrolyzing a cellulosic feedstock, said method comprising the step of incubating a medium comprising said cellulosic feedstock with Clostridium thermocellum, wherein said step of incubating is performed in two stages, wherein:
a. the pH of said medium is maintained at a value consistent with bacterial replication and/or metabolism during the first stage; and
b. the pH of said medium is not maintained at said level during the second stage, thereby hydrolyzing a cellulosic feedstock.

44. The method of claim 43, wherein the pH of said medium is uncontrolled during said second stage, or the pH of said medium is lowered to a level below 5.0 by addition of an acidifying agent during said second stage.

45. The method of claim 43, wherein said cellulolytic bacterium has been expanded on a medium containing cellulose and/or cellulosic biomass as the major energy source, prior to its introduction into said cellulose hydrolysis apparatus.

46. The method of claim 43, wherein the step of incubating is performed for at least 72 hours, and the cellulose concentration is at least 80 grams per liter of medium.

47. A product of a cellulose hydrolysis process utilizing the method of claim 43.


SEQUENCE LISTING (18 sequences)

SEQ ID NO: 1 - DNA, 6577 bp, Clostridium thermocellum
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatggcaaagataactttcccaaaagatttcatatggggttctgcaacagcagcatatcagattgaaggtgcatacaacgaagacggcaaaggtgaatctatatgggaccgtttttcccacacgccaggaaatatagcagacggacataccggcgatgttgcatgcgaccactatcatcgttatgaagaagatatcaaaataatgaaagaaatcggtattaaatcatacaggttttccatctcatggcccagaatctttcctgaaggaacaggtaaattaaatcaaaagggactggatttttacaaaaggctcacaaatctgcttctggaaaacggaattatgcctgcaatcactctttatcactgggaccttccccaaaagcttcaggataaaggcggatggaaaaaccgggacaccaccgattattttacagaatactctgaagtaatatttaaaaatctcggagatatcgttccaatatggtttactcacaatgaacccggtgttgtttctttgcttggccactttttaggaattcatgcccctgggataaaagacctccgcacttcattggaagtctcgcacaatcttcttttgtcccacggcaaggccgtgaaactgtttagagaaatgaatattgacgcccaaattggaatagctctcaatttatcttaccattatcccgcatccgaaaaagctgaggatattgaagcagcggaattgtcattttctctggcgggaaggtggtatctggatcctgtgctaaaaggccggtatcctgaaaacgcattgaaactttataaaaagaagggtattgagctttctttccctgaagatgacctgaaacttatcagtcagccaatagacttcatagcattcaacaattattcttcggaatttataaaatatgatccgtccagtgagtcaggtttttcacctgcaaactccatattagaaaagttcgaaaaaacagatatgggctggatcatatatcctgaaggcttgtatgatctgcttatgctccttgacagggattatggaaagccaaacattgttatcagcgaaaacggagccgccttcaaagatgaaataggtagcaacggaaagatagaagacacaaagagaatccaatatcttaaagattatctgacccaggctcacagggcaattcaggacggtgtaaacttaaaagcatactacttgtggtcgcttttggacaactttgaatgggcttacgggtacaacaagagattcggaatcgttcacgtaaattttgatacgttggaaagaaaaataaaggatagcggctactggtacaaagaagtaatcaaaaacaacggtttcctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 2 - protein, 456 aa, Clostridium thermocellum
MAKITFPKDFIWGSATAAYQIEGAYNEDGKGESIWDRFSHTPGNIADGHTGDVACDHYHRYEEDIKIMKEIGIKSYRFSISWPRIFPEGTGKLNQKGLDFYKRLTNLLLENGIMPAITLYHWDLPQKLQDKGGWKNRDTTDYFTEYSEVIFKNLGDIVPIWFTHNEPGVVSLLGHFLGIHAPGIKDLRTSLEVSHNLLLSHGKAVKLFREMNIDAQIGIALNLSYHYPASEKAEDIEAAELSFSLAGRWYLDPVLKGRYPENALKLYKKKGIELSFPEDDLKLISQPIDFIAFNNYSSEFIKYDPSSESGFSPANSILEKFEKTDMGWIIYPEGLYDLLMLLDRDYGKPNIVISENGAAFKDEIGSNGKIEDTKRIQYLKDYLTQAHRAIQDGVNLKAYYLWSLLDNFEWAYGYNKRFGIVHVNFDTLERKIKDSGYWYKEVIKNNGFLEHHHHHH

SEQ ID NO: 3 - protein, 471 aa, Clostridium thermocellum
MFPLGYNYIITLFANNILKGVVNMSKITFPKDFIWGSATAAYQIEGAYNEDGKGESIWDRFSHTPGNIADGHTGDVACDHYHRYEEDIKIMKEIGIKSYRFSISWPRIFPEGTGKLNQKGLDFYKRLTNLLLENGIMPAITLYHWDLPQKLQDKGGWKNRDTTDYFTEYSEVIFKNLGDIVPIWFTHNEPGVVSLLGHFLGIHAPGIKDLRTSLEVSHNLLLSHGKAVKLFREMNIDAQIGIALNLSYHYPASEKAEDIEAAELSFSLAGRWYLDPVLKGRYPENALKLYKKKGIELSFPEDDLKLISQPIDFIAFNNYSSEFIKYDPSSESGFSPANSILEKFEKTDMGWIIYPEGLYDLLMLLDRDYGKPNIVISENGAAFKDEIGSNGKIEDTKRIQYLKDYLTQAHRAIQDGVNLKAYYLWSLLDNFEWAYGYNKRFGIVHVNFDTLERKIKDSGYWYKEVIKNNGF

SEQ ID NO: 4 - DNA, 1416 bp, Clostridium thermocellum
atgtttcctctaggttataattatattattacactgtttgcaaataatatcttaaagggtgtggtaaacatgtcaaagataactttcccaaaagatttcatatggggttctgcaacagcagcatatcagattgaaggtgcatacaacgaagacggcaaaggtgaatctatatgggaccgtttttcccacacgccaggaaatatagcagacggacataccggcgatgttgcatgcgaccactatcatcgttatgaagaagatatcaaaataatgaaagaaatcggtattaaatcatacaggttttccatctcatggcccagaatctttcctgaaggaacaggtaaattaaatcaaaagggactggatttttacaaaaggctcacaaatctgcttctggaaaacggaattatgcctgcaatcactctttatcactgggaccttccccaaaagcttcaggataaaggcggatggaaaaaccgggacaccaccgattattttacagaatactctgaagtaatatttaaaaatctcggagatatcgttccaatatggtttactcacaatgaacccggtgttgtttctttgcttggccactttttaggaattcatgcccctgggataaaagacctccgcacttcattggaagtctcgcacaatcttcttttgtcccacggcaaggccgtgaaactgtttagagaaatgaatattgacgcccaaattggaatagctctcaatttatcttaccattatcccgcatccgaaaaagctgaggatattgaagcagcggaattgtcattttctctggcgggaaggtggtatctggatcctgtgctaaaaggccggtatcctgaaaacgcattgaaactttataaaaagaagggtattgagctttctttccctgaagatgacctgaaacttatcagtcagccaatagacttcatagcattcaacaattattcttcggaatttataaaatatgatccgtccagtgagtcaggtttttcacctgcaaactccatattagaaaagttcgaaaaaacagatatgggctggatcatatatcctgaaggcttgtatgatctgcttatgctccttgacagggattatggaaagccaaacattgttatcagcgaaaacggagccgccttcaaagatgaaataggtagcaacggaaagatagaagacacaaagagaatccaatatcttaaagattatctgacccaggctcacagggcaattcaggacggtgtaaacttaaaagcatactacttgtggtcgcttttggacaactttgaatgggcttacgggtacaacaagagattcggaatcgttcacgtaaattttgatacgttggaaagaaaaataaaggatagcggctactggtacaaagaagtaatcaaaaacaacggtttttaa

SEQ ID NO: 5 - protein, 754 aa, Clostridium thermocellum
MAVDIKKIIKQMTLEEKAGLCSGLDFWHTKPVERLGIPSIMMTDGPHGLRKQREDAEIADINNSVPATCFPSAAGLACSWDRELVERVGAALGEECQAENVSILLGPGANIKRSPLCGRNFEYFPEDPYLSSELAASHIKGVQSQGVGACLKHFAANNQEHRRMTVDTIVDERTLREIYFASFENAVKKARPWVVMCAYNKLNGEYCSENRYLLTEVLKNEWMHDGFVVSDWGAVNDRVSGLDAGLDLEMPTSHGITDKKIVEAVKSGKLSENILNRAVERILKVIIMALENKKENAQYEQDAHHRLARQAAAESMVLLKNEDDVLPLKKSGTIALIGAFVKKPRYQGSGSSHITPTRLDDIYEEIKKAGADKVNLVYSEGYRLENDGIDEELINEAKKAASSSDVAVVFAGLPDEYESEGFDRTHMSIPENQNRLIEAVAEVQSNIVVVLLNGSPVEMPWIDKVKSVLEAYLGGQALGGRWRMCYSVKSIVGKLAETFPVKLSHNPSYLNFPGEDDRVEYKEGLFVGYRYYDTKGIEPLFPFGHGLSYTKFEYSDISVDKKDVSDNSIINVSVKVKNVGKMAGKEIVQLYVKDVKSSVRRPEKELKGFEKVFLNPGEEKTVTFTLDKRAFAYYNTQIKDWHVESGEFLILIGRSSRDIVLKESVRVNSTVKIRKRFTVNSAVEDVMSDSSAAAVLGPVLKEITDALQIDMDNAHDMMAANIKNMPLRSLVGYSQGRLSEEMLEELVDKINNVE

SEQ ID NO: 6 - DNA, 2894 bp, Clostridium thermocellum
aacaggataaagcttaccggtgagataaacaacattccgtttacgtttacaggcacgggttacagttttgtagttgaaagcttcaaaataaaagtaaaattcctcaaaccgggcacaatagtatttgacggggcagattccagactaagatacaattttaattacgttgacggagcaggaaacattcattcaaagagcgtggacaaacattttgacgacatgacggtgaatgtcacgatgaaggttgatataaactgatgggttgccaaaagacgatttaacatatatatgcacatataaacgcaaagcatgaggaggatagaaatggcggtagatatcaagaaaataataaagcagatgactttggaagaaaaagcagggttgtgctcgggactggatttttggcataccaagcctgttgagagactgggcattccttcaataatgatgactgacggacctcatggactgagaaagcagagggaagatgcagagattgcggacatcaacaacagcgttccagcaacctgttttccgtctgcagcaggtttggcatgttcctgggacagagaactggttgagagagtaggtgcagcactaggagaagaatgtcaggcggaaaatgtctcaatactgcttggaccaggtgcaaatataaagcgttcacctttgtgtggaagaaattttgaatattttcccgaagacccttatctttcgtcagagctggcggcaagccatataaaaggagttcaaagtcagggagtgggtgcatgtcttaaacattttgccgcaaacaaccaggaacaccggagaatgaccgttgataccattgtagatgaaagaacgttgagggaaatatattttgcaagctttgagaatgctgtaaaaaaagcacggccttgggtggttatgtgtgcatataacaagctcaacggtgaatattgttcggagaacagatatcttttgacggaagttttaaagaatgaatggatgcatgacggctttgtggtatccgactggggtgcggtaaatgacagggtcagcggcctggatgcaggtcttgacctggaaatgcccaccagtcatggtattacggataaaaagatagttgaagccgtaaaaagcggaaagctgtctgaaaatattttaaacagagctgtggaaagaattttgaaagtaattattatggcactggaaaacaaaaaagaaaacgcgcagtatgaacaagatgctcatcacagactggcaaggcaggctgcggccgaatcgatggttcttcttaaaaacgaggacgatgtgcttcctttaaaaaagagcggaaccatagctttgataggagcttttgtgaaaaaaccaagataccagggttcgggcagttctcatattaccccgacaagacttgatgatatttatgaagagataaaaaaggccggagccgacaaagtaaaccttgtatattcggaaggatacaggcttgaaaatgacggtattgatgaggaattgataaacgaagctaaaaaggcggcatcaagctcggatgttgcggtagtatttgcagggcttccggatgaatatgaatctgaaggatttgacagaactcacatgagtattccggaaaatcaaaacaggctgatagaagcggtggccgaagtccagagtaatattgttgtggtattgcttaacggctcaccggttgaaatgccgtggattgacaaggtaaaatccgtgcttgaagcttatcttggaggccaggcgctgggaggccgctggcggatgtgctattcggtgaagtcaatcgtcggaaaacttgcggagaccttcccggtgaaattaagccataatccgtcctatttgaattttcccggagaggatgaccgagtggagtataaagaagggttgtttgtcggatacagatattatgatacaaagggaattgagccattgttcccctttggtcacggacttagctataccaaatttgaatacagtgatatatcagtcgataaaaaagatgtttcggacaatagcatcataaatgtcagcgttaaagtcaaaaatgttggaaaaatggcaggaaaagaaattgtgcagctgtatgtaaaagatgtgaaaagcagcgtcagaagacctgagaaagagcttaaaggatttgaaaaggtcttccttaatccgggagaagaaaagacggttacatttactttggacaaaagggcttttgcatattacaatactcagattaaggactggcatgttgaaagcggagagtttctgatattaataggaaggtcctccagggacatagttttaaaagaatcagtgagagtaaattcaacggtgaagataagaaaaagattcacagtgaattcagcggttgaagatgtaatgtccgattcttcggctgcggccgttttagggcctgtactaaaagagataaccgatgcactgcagattgatatggacaatgctcatgacatgatggcggccaatataaagaatatgcctttgcgctcacttgtcggttactctcagggaaggttaagcgaagaaatgctggaggaactggttgacaaaataaacaacgtggaataaatgggtttggtgcagataaaaggtaatgccgaagtatataaaacttcgcattaccttttgtttaatttaagaaatggaataatattaaatatttttatgtaatatattaaaataaataattaaatataaagcaaaaaacttttgcatgtaaaaaacatgatttttaatgtacgttgtctaattgtggcagggtgaatacgggaaagaaggaggagatccggatgctgccggtgaatacatttctttatgcctttgttttaatagcttttttggcggcttttttaacaggaatagttctcattata

SEQ ID NO: 7 - protein, 795 aa, Lactobacillus casei
MGVVVSNFHLAKITAEEKVKLTSGKDFWTSEHLADKGIPSFRMSDGPHGLRYQALAADHLGINDSVPSTSFPTASASAAAWDPDLIQAMGKAIGLEAQSLGVDMVLGPGVNMKRNPLCGRNFEYFSEDPFLAGKLGAAWINGIQSQGIAACLKHFAANNQENDRLSSDSLVDPTALHEIYLEAFRIAVTESHPEAVMCSYNKINGTYASDNLYLMTQVLRQQFGFGGAVITDWGALNDKVAALNAGTDLEMPGDDHLFDGEALQAYQQGTLKLASLDRAVTKIAEIARKQRPKFQGSREQLLQANGQLAQKIAESAIVLLKNEAALLPLQATDTVAVIGELAKATRFQGAGSSHINASEIVSVLDGLKQKKVSFDYAAGYRLDDQDDSQATAEALALARNHDKVVFVAGLPDNYESEGFDRQNMALPKVQNDLLQAVTAVNPNVIVLLVAGAPVELPWVDQVKAVVNLSLGGERIGAAAANVLTGAVNPSGKLAESYPLKYQDVPSADVYDKNPRSVPYVESTYIGYRYYDKAKVPVAFPFGFGLSYTSFALKNIQLSSDHVTDDQPLTISLQVTNTGQVDGAEVVQVYVQEQQPRPLRPEKSLKAFKKVFVKAGQTVNVALELKAQAFKEWREQTQTWVLPEAQKAIAVGTSVTNIDAVLPVSFTGETFNNFATIPNWYTTLSGKPSVQDFEQLTDQKVPAPHEFVPGEFTRLNTPREMKKHSLLLRLVAWITVKIRTKDYIDKQGPEAKFQQAIVLDTPLIRLAQQASGALKLSMVDRLVAAANHQYVKMIFR

SEQ ID NO: 8 - protein, 764 aa, Bacteroides thetaiotaomicron
MNMKFKATLLGLSIAAVLPTMNMAQTPVYLDTSKPIEERVKDALSRMTLEEKVKMTHAQSKFSSPGVPRLGIPEVWATDGPHGIRPEVLWDEWDQAGWTNDSCIAYPALTCLSATWNPEMSYLYGKSIGEEARYRKKDILLGPGVNIYRTPLNGRNFEYMGEDPYLSSMMVVPYIKGVQENGVAACVKHYALNNQEFNRHTTNVHLSDRALYEIYLPAFKAAVQEGGAWAIMGAYNLYSFSEDTDSGKLYKTQHACHNKRLLQDILRKEWGFDGVVVSDWGGVHDTFQAISNGLDMEFGSWTNGLSAGTRNAYDNYYLAHPYLKLIQDGTVGTKELDEKVSNILRLIFRTSMNPHKPFGSLASPEHGQAGRKIGEEGIVLLQNKDNVLPIDLKKARKIAVIGENAIKMMTVGGGSSSLKVKYEISPLDGLKNRVGSQAEVLYVRGYVGDPTGEYNGVQTGQDLKDDRSEDELLAEAVEVSKDADYVIFFGGLNKSNHQDCEDSDRASLGLPYAQDRVIGELAKVNKNLIVVNISGNAVAMPWVNEVPAIVQGWFLGSEAGTALASVLLGDANPSGKLPFTFPARLEDVGAHKLGEYPGNKEELAHSKNNGDTINEIYREDIFVGYRWADKEKIKPLFPFGHGLSYTTFAYGKPSADKKVMTADDTISFTINVKNTGTREGQEVIQLYVSDKKSSLPRPVKELKGFKKVKLAPGEEKAVTLTIDKKALSFFDDVKHEWMTEPGKFEAVIGTSSRDIKGIVPFELR

SEQ ID NO: 9 - protein, 811 aa, methanogenic Archaeon RC-I
MKEFIKNIIASMTLEEKASLCSGLDMFRLKGIARLGIPSILLSDGPHGLRKPVFDPDHLGIGQSIPSTCFPTASAMASSWDRNLLYEIGYALGEECLHEDVAVILGPGANIKRSPLCGRNFEYFSEDPYLTGELAASMIEGIQATGVGASLKHFAVNNQEYRRMTIDAIVDERTLREIYLAGFEMAVKQSRPRTVMCSYNKVNGVYASEHERLLTDILRKEWGYEGLVMTDWGACDDRVAGLKAGQDLEMPSSFGVNDAKIVKAVRDGTLSEAVLDEAVERVLELVYDAVENRLPDYCYEKRTHHVMARQAAAESMVLLKNDGILPLKKGMKIAVIGAFAIHPRYQGNGSSQVNPCRLEKAYCELCTYTSEITFARGYDLRSDVPDERLITEACNIARGAEVAIIFAGLPDSYETEGMDREHMRMPESHNELIRRVAEANPGTVVVLANGAPVEMPWLGNVKAVLEGYLGGEAAGGAAADLLFGMVNPSGKLAETFALQLDDYPSANYFPSGPRTVEYREGLYVGYRYFDTAKKAVLFPFGHGLSYTSFEYSDMEVSASQIKDDEELKVGVLVKNTGDVAGAEVIQLYVRDVESTIFRPEKELKGFDKVFLQPGEMTRVEFTLDRRSFAYYNVETADWHVESGDFEILIGSSSADIRARETIWVESTRPGVSVPDLRSTAGIYYNLPPGNLVVDDESFKAIYGETLPSNIVLEGEPYTINSTLGEVKETFFGRIFFELVHNSARNTLPEAEDGEMQKIMTEKMLEDLPLRNVLTFSEGKINEQMLEGLLLLINRKPAQGLVRLSTALLKIG

SEQ ID NO: 10 - protein, 782 aa, Dictyoglomus thermophilum H-6-12
MKLEYKIPYRIERGEQTNFSPLFIRIISQGGKQMEKDIKKLISQMTLEEKASLCSGLDFWHTKPIERLGIPSIRMSDGPHGLRKEETMFSKTVPATCFPTAVTIAASWDKLLAEKMGKAIGEECQAENVQILLGPGINMKRSPLCGRNFEYYSEDPILAGELAAHFIKGVQSQGVGTSLKHFAANNQEHRRLTVDAIIDERTLREIYLTAFEKAVKEAKPWTVMCSYNKVNGTYASENEFLLTKVLREEWGFEGFVVSDWGAVNDRVKGLAAGLDLQMPYDGGNGDKKIIEAVKSGKLPEEVLDRAVERILKIVFKAIENKKENATYDKEAHHKLAREIARECFVLLKNENNILPLKKEGKIALIGAFAKKPQIQGGGSAHVNPTRVDDAVEEIKKLVGDKVEILYADGYHIEKDDVDEKLIEEAKEIAKKADVVVIFAGLPERYESEGFDRPHMKMPESHNRLIEEVAKVNSNLVVVLSNGAPIEMPWVDKPKAILETYRGGQAWGGAVADVLFGVVNPSGKLPESFPKKLSDNPSYLFFPGEDDRSEYREGIFIGYRYYDKKEMEVLFPFGYGLSYTTFEYSDLKLDKKEMKDDEVLKVSVKVKNTGKVKGKEIVQLYVRDVKSNYIRPEKELKGFEKVELEPGEEKEVVFYLDKRAFAFYNIDIKDWYVEDGEFEILIGKSSRDIVLKDKVFVKSTTKIKRHYHINSTIGDIMSDPEASAKFKHILEQFASAFPAFSSEEAIMNFAEMMKYMPLRNLIHFGQGKFTEEMLENLLKEINS

SEQ ID NO: 11 - protein, 448 aa, Streptomyces coelicolor A3(2)
MATDDAMPAKPMPRFPDGFLWGVSTSAHQIEGAAGLRGPSVWDAFTAEPGRVRDGSTAAVACDHYHRYREDVALLAGLGVDAYRFSVSWPRVDSPGGLDFYDRLVDELCAAGVRPVPTLFHWDLPAGLDWLERDTAARFAEYVSLVAERLGDRVGKWITLNEPAEHTLLGHALGVHAPGRELLFDALPVAHHQLLAHGLAVRALRAAGATDVGIANSHGPTWPASDDPADREAADFYDLLLNRMFADPLLTGRYPEGVGALMPGSAERVEADLEVIAEPLDWYGVNYYAPTRVGAPQGAEIEFGGVTLPAELPFSVRRIEGRPVTDFGWPVVPEGLTELLTGFRDRYGDRLPPVVITENGCSYEGLDDHDRIAYLDGHVRALHRAIEAGVDVRGYFVWSLLDNFEWAEGYARRFGLVHVDFTTLARTPKASYGWFRDLLDGQRRVPVG

SEQ ID NO: 12 - protein, 860 aa, Aspergillus niger
MRFTSIEAVALTAVSLASADELAYSPPYYPSPWANGQGDWAEAYQRAVDIVSQMTLAEKVNLTTGTGWELELCVGQTGGVPRLGIPGMCAQDSPLGVRDSDYNSAFPAGVNVAATWDKNLAYLRGQAMGQEFSDKGADIQLGPAAGPLGRSPDGGRNWEGFSPDPALSGVLFAETIKGIQDAGVVATAKHYIAYEQEHFRQAPEAQGYGFNITESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTISVLNGTVPQWRVDDMAVRIMAAYYKVGRDRLWTPPNFSSWTRDEYGFKYYYVSEGPYEKVNQFVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVIIHSVGPVLVNEWYDNPNVTAILWGGLPGQESGNSLADVLYGRVNPGAKSPFTWGKTREAYQDYLYTEPNNGNGAPQEDFVEGVFIDYRGFDKRNETPIYEFGYGLSYTTFNYSNLQVEVLSAPAYEPASGETEAAPTFGEVGNASDYLYPDGLQRITKFIYPWLNSTDLEASSGDASYGQDASDYLPEGATDGSAQPILPAGGGAGGNPRLYDELIRVSVTIKNTGKVAGDEVPQLYVSLGGPNEPKIVLRQFERITLQPSKETQWSTTLTRRDLANWNVETQDWEITSYPKMVFAGSSSRKLPLRASLPTVH

SEQ ID NO: 13 - protein, 723 aa, Listeria monocytogenes
MKQEKVQDLVNQMTLDEKIAQCLQLSPFLFKGTNKNAELTGPLLQEMKLTDAHTENAGSVLGSSSALDMIGIQEAYLKTNRLGIPLVFMADVIHGYKTVFPIPLALGCSFDRETVRVMAEVSALEATADGHHVTFSPMLDLVRDPRWGRVMESTGEDPFLNSELGKAMVDGYQGDASKLHENLEQMAACVKHFAAYGAAEAGLEYNTVNMSTRELYQNYLPAYNAAIQAGAKLVMTAFNVVDGIPATMNKWLNRDVLRDEMGFDGVLISDWGAVAEVINHGTARNPKEAAQFSMDAGVDLEMMTTCYIHELKGLIEEGKLSESLLDEAVLRMLTLKNDLGLFEDPYRGLKNNDRTKDILTDDSRGKARAAGIESAVLLENKNRLLPLAKEAKIALVGPLATSPDILGGWNVYGEEKDGINVETGLREVFETVEVVSTEYTELSEEDKVAVKAAVENMDVVVLALGEKNEWGGEAGSLATIRLPEAQYELAKFVQTLGKPVVITLFNGRPLEVKELAESSDALLELWFPGTEAGRVTADLLSGASNPSGKLSMSFPQTTGQIPVYYNHLRTGRPQTPENKGERYVSHYLDIPNEPFYPFGYGKSYSEFELKTSSLPKELNLGESLHVEVTIKNISDIAGKEVIQVYLQDVTASISRPVKELKAFEKVALQAGEEKTVRFELTSEAFSFYNQQLEKVQEPGLHRVFVGTSSEDVDVFEVEVGGYV

SEQ ID NO: 14 - protein, 739 aa, Sorangium cellulosum
MRLTRYATWIMAAAVVLAPAACGDDTSDNPTGSGASSGAGAGSGASSGAGAGSGSGSGSGSGSGAGAGSSGSGSGTGGGGTGGSDSTGGGGSGAGDSVGLLPCDNGWPEVRSAIAQDPAIEAAIAELLGKMKVEEKVGQMVQAEIQKITPAEVKQYNIGSVLNGGGSWPGKNKNATAADWVKLADDFYNASVDTSGGRVGIPIIWGIDAVHGNNNVRGATLFPHNIGLGAAHDPDLLERIGAATAKEVLATGLDWTFAPTLATVRDDRWGRTYEGYSEDPEIVNAYGGRIVQGIQGAANSPDLLGATRVIATAKHFIGDGGTDKGDDQGNNLASDTELCTIHAQGYLSAIPAGAQTVMASYNSIRGQKMHGKGDLLTGVLKDKFHFDGFVIGDWNGHGQVSGCTNSSCAASINAGVDMIMVPDDWKAFYENTLSQVKGGQISMARVDDAVTRILRVKMRAGLLGPKKTKQAPSKRMFAGDQSVLGQAEHRALAREAVRKSLVLLKNARGVLPLAASAKVLVAGKSADSISNQSGGWSRTWQGTELTNADFPGATSIFKGIQDLVSAGGGQATLSADGSGASSGSFDAAIVVIGETPYAEMQGDIQVATDDTPHAKTLEHAAYHPEDLQVLQAIRTAKSDLPIVTVFLSGRPLYVNKELNRSDAFVAAWLPGSEGGGVADVLFGKQQFQGKLSFSWPATECQRVNRGDDGALFPYGFGLTTENKETPAALPEPSSGGGCN

SEQ ID NO: 15 - protein, 723 aa, Xanthomonas campestris
MAADRIDSLIARMTVEEKVGQLGVFADMVRPFAPDVNPEANVLNADEVLQQVRQGRVGSLFNGVGAALGVQIQKVAVEESRLGIPVILAADVIHGMRTVFPIPLGEAASFEPELAERTARATAIEATAAGLHWTYAPAVDIARDQRWGRGAEGAGEDVVLGMAFAAARVRGFQGSDLKADDCLLATPKHFAAYGAVAAGMEYNTVDIAPQTLRDVHLPPFKAAFDAGALTVMSSFNDINGVPASANHELLTEILRGEWQFPGVVISDYTADMELIAHGYAADERDATKKAFLAGLDLSMQSGFYAAHLPSLVESGEVPMATLDASVRRILQLKEAIGLFDNPYRSLDPAREADTAHLPAHDALSRDAARRSIVLLKNEGDLLPLKKSGQNIALIGPFVQDRENIEGCWTLFGDKERYVTLEQGVRAVVAADNLSVVAGCGLEEPLPGGISAAIDAAQAADVVVLALGEPQRFSGEAQSRTEITLPPAQQALAEAVAATGTPMVILLRNGRALALSGAVRDADAIAVTWYLGTQTGTGVADVLFGDYNPSARLPISFPQVTGQQPYFYNHLRTGRPELPTLSEYKARWREMPNEPLYPFGHGLSYTTFAYAAPQLSTTQLGWEQTLTITTRVTNTGTVAGEEVVQLYVHDRVASRVRPVRELKGFRKVLLQPGESAEVVFTLQRDALAFTNHKGVFGAEPGLFDVWVCASAKSGEPVSFELLDR

SEQ ID NO: 16 - protein, 768 aa, Pectobacterium atrosepticum
MKWLTSLTIAIGLACNPTFAQELTSVPASISPSHQQRDAFVTDLLKKMTLEEKIGQLRLISVGTDNPKEVIREMIRNGQVGAIFNTVTRQDIRAMQDQVMQLSRLKIPLFFAYDVVHGQRTIFPIALGLASSWDMDAIAKSARVAAYEATEDGLNMTWAPMVDITRDPRWGRVSEGFGEDTWLTSKIAGVVVKSFQGDDVTGRHSLMTSVKHYALYGAVEGGRDYNTVDMSPQRMFQDYMPPYKAAIDAGSSGVMVALNSINGTPATANSWLLKEVLRDQWNFKGITITDHGAIKELIKHGVASDPRDASRLALKSGIGMSMSDEYFVRYLPELVKSGAVSVQEIDDACRQVLNVKYDMGLFEDPYRHLGIASSDPVDTNAESRLHRLDARDVARKSLVLLKNRLQTLPLKKQGTIAVVGPLADSQRDTMGSWSAAGVTKQTITVYQGLKNAVGDKATILYAKGANVSNHKGIIDFLNQYEDAVQVDKRPPQVMIDEAVEAAKKADVVVAVVGEAAGMAHEASSRSNIDLPQGQRDLIAALKATGKPLVLVLMNGRPLALVREDQQADALLETWFSGTEGGNAIADVLFGDYNPSGKLPMSFPRSVGQIPIYYNNLPSGRPYTPENPGKYTSHYYDEANGPLYPFGYGLSYTTFSVSDVRLSSQTMKRNGTINASVTVKNTGSRAGETVVQLYLHDVVASISRPLKELRGFEKVMLQPGESRTVTFTLDQDALKFYNARMQQVVEPGKFDVMIGLDSQRVKSDSFTLL

SEQ ID NO: 17 - protein, 714 aa, Lactococcus lactis
MKEKELWNLFQSMTLAEKLGQMTQTTGEYFVGKEFAEELVVTGPAMEEMGFDSENIHRVGSVLGVSSSQAINAVQRAYLEKSRLGIPLMFMHDAIHGYRTIFPIPLALASTFNTKLIEKVGHLTGQELVATGIKVNFSPMVDLVRDARWGRVMESFGEDSILSGRLGRAMIEGYQGSADGTIGKQNVIACLKHFVAYGAPDGGRDYASVDMSKKELFGFYAKSFEIALMAFPRMVMASFNSFNGEPVTASSYLLEEVLRQKFTFTDLLISDWGAVSELKNHGIAANDKEAGYLALNAGIEIEMVSNTFLKYGEEYLSRKPSLIEKINQAVWDFLNLKNEFGLFEHPYVDENEEQKVIRSKKIVNAACEISEASCVLLKNENDLLPLKKEDTLLIIGPFAKTQDLLGNWNCKGKAAETISVEQGFKNLASNVHAYKYLDEVPEELLEKSDNVLVTIGERWDKSGEGHSSVDLEIDTAQQSLIYDLKAMGKKVIGLGFSGRPMALGAVIDDLDALLWTWYLGNEAGNAIANLILGIKSPTGRLAMSFPRVSAQVPLRYNELGSGRPANDSTYSSRYQDLPIGPLFPFGYGLRYGRVKIAKVELSSSVISDDNPLNISLELTNNSEYDTSESIILFMEDSISKIVRPVRELIDFQCIALKKGENRKVEFVVQTSDLAYINNEEKKVFENGTINFYINDLNKKVANVEVQNRREGIYK

SEQ ID NO: 18 - protein, 797 aa, Aspergillus fumigatus
MVGTFLSKSLLLLQLVSILSTGQAASTPLYKDPSAPVDDRVRDLIGRMTIEDKMAQLMQGDITNWMNSTSGAFNYTGLVENMKMKAGSFYVGYPVPWDWIATNVKRAQDYLMQNTTLGIPALVQTEGIHGFLIGNATIFNSPIAYGCSFNREVRISHLVAWGKSNRELQLVQEMAKYVAQEARTLGVTQLFAPVVDLARELRFGRVEETFGEDPYLSGEMGYSYVKGLQSLNVSSMVKHFIGFSQPEQGINTAPVHGGERYLRTTWFPSFKRAIIDAGAWSIMSAYHSYDGIPAVSDYHTLTEILRGEWGYDYFVMSDAGGTDRLCSAFKLCRSNPIDMEAVTLQVLPAGNDVEMGGGSFNFQKIPELVKAGKLDIKTVDTAVSRVLRAKFEMGLFENPYPAAPKSQWNNLIHSKEAVKLARQLDKESIVLLENHNNTLPLKKKGDIAVIGPMAHGFMNYGDYVVHKSQYRGVTPLDGIKAAVGKKANIHYAKGCERWSNDQSGFAEAVEAAKKSNVAIVVVGTWSRDQMELWQGLNATTGEHVDVNDLSLVGAQAPLIKAIVDTGVPTIVVLSSGKPVTETWLSNSTAALVQQFYPSEEGGNALADVLFGDYNPSGKLSVSFPSYVGDLPIYYDYLNSARSIGDSGYELPNGTFVFGHQYVFGSPLPWYPFGYGKSYSTFEYGPVTVDKANVTASDTVTVSVDVTNTHKSMDGTEVVQVYIQDEISSVVVPNRQLKGFEKVVIPAKKTKTVKIKIKVQDLGLWNSAMKYVVEPGAFTALVGSSSADIRGNATFYVQ